SEQUENCE LISTING (1 sequence)

SEQ ID NO: 1 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

(19) United States
(12) Patent Application Publication
(10) Pub. No.: US 2021/0188933 A1
(43) Pub. Date: Jun. 24, 2021

(54) METHODS OF TREATING PANCYTOPENIA

(71) Applicant: NantCell, Inc, Culver City, CA (US)

(72) Inventors: John Lee, Culver City, CA (US); Patrick Soon-Shiong, Culver City, CA (US)

(21) Appl. No.: 16/817,529

(22) Filed: Mar. 12, 2020

Related U.S. Application Data
(63) Continuation-in-part of application No. 16/719,509, filed on Dec. 18, 2019, now abandoned.

(51) Int. Cl. C07K 14/54; C07K 14/715; C12N 5/078

(52) U.S. Cl. CPC C07K 14/5443; A61P 7/00; C12N 5/0634; C07K 14/7155

(57) ABSTRACT

Pancytopenia, in particular lymphopenia is treated with compositions which include an IL-15 superagonist, IL-15, an IL-15 mutant, IL-15:IL-15 receptor alpha (IL-15Rα) complex or combinations thereof. Administration of preferred compositions results in the increase of blood cells, in particular, lymphocytes in subjects in need of such treatment.

This application is a continuation-in-part of U.S. application Ser. No. 16/719,509, filed Dec. 18, 2019 which is herein incorporated by reference in its entirety.

### FIELD

The present disclosure provides embodiments directed to the prevention and treatment of a subject suffering from or susceptible to pancytopenia. Various embodiments comprise administering interleukin 15 (IL-15), an IL-15 superagonist, an IL-15 mutant, or combinations thereof. Preferred compositions include a therapeutically effective amount of IL-15, IL-15 superagonist, IL-15 mutant, or combinations thereof.

### INCORPORATION BY REFERENCE OF SEQUENCE LISTING

The instant application contains a Sequence Listing which has been submitted electronically in ASCII format and is hereby incorporated by reference in its entirety. Said ASCII copy, created on Dec. 17, 2019, is named 055537_507F01US_PAT005023.US0001_ST25.txt and is 1,693 bytes in size.

### BACKGROUND

Prolonged pancytopenia common follows intensive chemotherapy regimens, myeloablative & reduced intensity regimens for hematopoietic cell transplantation (HCT), and acute exposure to ionizing radiation. Prolonged lymphopenia and/or neutropenia significantly increases infection risk, morbidity, and mortality.

Oelert & al. (2010) Blood 115(11):2196-202 report that CD8 T-cell tolerance is lost after splenocytes are transferred into irradiated lymphopenic mice, and that this phenomenon is dependent on the presence of IL-15. They found that when mastocytoma cells were inplanted in mice, tumors grew out in all mice, and injection of an IL-15/IL-15Rα complex did not break tolerance of Kb-tolerant cells in nonirradiated Rag2−/− mice.

Novel therapies to ameliorate prolonged pancytopenia following high dose chemotherapy and/or radiation are needed.

### SUMMARY

In one aspect, methods and compositions are provided to prevent and/or treat a subject suffering from or susceptible to pancytopenia. Methods of preventing and/or treating pancytopenia comprise administering IL-15, IL-15 superagonist, an IL-15 mutant, or combinations thereof. Compositions include a therapeutically effective amount of IL-15, IL-15 superagonist, an IL-15 mutant, or combinations thereof. Accordingly, in certain embodiments, a method of preventing or treating pancytopenia in a subject comprises administering to the subject a composition comprising a therapeutically effective amount of IL-15, an IL-15 superagonist, an IL-15 mutant, or combinations thereof. Preferably, the IL-15, IL-15 superagonist, IL-15 mutant, or combinations thereof increases the subject's blood cell count. In certain embodiments, the IL-15 superagonist may be an IL-15:IL-15Ra complex.

In certain embodiments, pancytopenia is induced by one or more factors comprising: anemia, cancer, infectious agents, immune disorders, genetic disorders, bone marrow disorders, hypersplenism, organ injury, kidney disease, transplantations, myelodysplastic syndromes, gastrointestinal disorders, chronic virus infection, recurrent virus infection, age related, drug-induced, chemotherapy, surgery, radiation therapy, steroid therapy, splenic sequestration, toxins, chemical exposure, radiation, or combinations thereof.

In certain embodiments, the method further comprises administering one or more one or more chemotherapeutic agents, compounds, cytokine antagonists, cytokine receptor antagonists, cytokines, chemokines, growth factors, pluripotent granulocyte colony-stimulating factor (“hpG-CSF”), erythropoietin (EPO), lymphopoietin, thrombopoietin, hormones, adoptive cell therapy, anti-viral agents, anti-microbial agents, anti-fungal agents, anti-parasitic agents, viral entry receptor antagonists, checkpoint inhibitors, adjuvants, or combinations thereof.

In certain embodiments the pancytopenia is lymphopenia.

In certain embodiments, a method of preventing or reducing lymphopenia in a subject in need thereof is provided that comprises administering to the subject a composition comprising a therapeutically effective amount of IL-15, an IL-15 superagonist, an IL-15 mutant, or combinations thereof. Preferably, the IL-15, IL-15 superagonist, IL-15 mutant, or combinations thereof increases lymphocyte counts in peripheral tissues, lymphocyte activity, or a combination thereof. In certain embodiments, the IL-15 superagonist may be an IL-15:IL-15Rα complex.

In certain embodiments, the method of preventing or treating pancytopenia or lymphopenia further comprises administering one or more chemotherapeutic agents, compounds, cytokine antagonists, cytokine receptor antagonists, cytokines, chemokines, growth factors, hpG-CSF, EPO, lymphopoietin, thrombopoietin, hormones, adoptive cell therapy, anti-viral agents, viral entry receptor antagonists, checkpoint inhibitors, adjuvants, or combinations thereof.

In certain embodiments, the cytokines are T helper 1 (T_{H}1)-inducing cytokines. In certain embodiments, the T_{H}1-inducing cytokines comprise IL-2, IL-10, IL-12, IL-18, IL-27, tumor necrosis factor-alpha (TNFα), tumor necrosis factor-beta (TNFβ), interferons, or combinations thereof.

In certain embodiments the cytokines are T helper 2 (T_{H}2)-inducing cytokines. In certain embodiments the T helper 2 (T_{H}2)-inducing cytokines comprise IL-4, IL-5, IL-6, IL-9, IL-13, IL-19, IL-25, IL-31, IL-33, interferons, or combinations thereof.

In certain embodiments, the adoptive cell therapy comprises administration of lymphocytes, hematopoietic stem cells, or combinations thereof. In certain embodiments, administration of hematopoietic stem cells further comprises administering one or more of the following growth factors: stem cell factor (SCF), also known as the c-kit ligand or mast cell growth factor, Flt-3 ligand (Flt-3L), interleukin-6 (IL-6), interleukin-3 (IL-3), interleukin-7 (IL-7), interleukin-11 (IL-11), thrombopoietin (TPO), granulocyte-macrophage colony stimulating factor (GM-CSF), G-CSF, angiopoietin-like proteins (Angptls) (Angptl2, Angptl3, Angptl5, Angptl7, & Mfap4), insulin growth factor-2 (IFG-2), IGFBP2, Wnt3a, and fibroblast growth factor-1 (FGF-1).

In certain embodiments, the subject is suffering from a disease or disorder associate with lymphopenia. In certain embodiments, the disease or disorder associate with lymphopenia comprises: cancer, infectious agents, immune disorders, genetic disorders, bone marrow disorders, hypersplenism, organ injury, kidney disease, transplantations, myelodysplastic syndromes, gastrointestinal disorders, chronic virus infection, recurrent virus infection, age related lymphopenia or combinations thereof. Genetic disorders comprise: Wiskott-Aldrich syndrome, adenosine deaminase deficiency, severe combined immunodeficiency disorder, purine nucleoside phosphorylase deficiency, ataxia-telangiectasia or DiGeorge anomaly.

In certain embodiments, the subject is undergoing or has undergone a treatment or therapy resulting in lymphopenia. The treatment or therapy comprising chemotherapy, surgery, radiation therapy, steroid therapy, drug therapy or combinations thereof. In certain embodiments the lymphopenia is due to aging of the subject.

In certain embodiments, a method of preventing or treating lymphopenia in a subject infected with human immunodeficiency virus (HIV) is provided that comprises administering to the subject a composition comprising a therapeutically effective amount of IL-15, an IL-15 superagonist, an IL-15 mutant, or combinations thereof. Preferably, the IL-15, IL-15 superagonist, IL-15 mutant, or combinations thereof, increase lymphocyte counts in peripheral tissues, lymphocyte activity, or a combination thereof. In certain embodiments, the IL-15 superagonist may be an IL-15:IL-15Rα complex. In certain embodiments, the method further comprises administering one or more chemotherapeutic agents, compounds, cytokine antagonists, cytokine receptor antagonists, cytokines, chemokines, chemokine receptor antagonists, growth factors, hpG-CSF, EPO, lymphopoietin, thrombopoietin, hormones, adoptive cell therapy, anti-viral agents, viral entry receptor antagonists, checkpoint inhibitors, adjuvants, or combinations thereof.

In certain embodiments, the antiviral agents comprise a therapeutically effective amount of antibodies, aptamers, adjuvants, anti-sense oligonucleotides, ribavirin, protease inhibitors, helicase inhibitors, integrase inhibitors, fusion inhibitors, polymerase inhibitors, helicase inhibitors, neuraminidase inhibitors, nucleoside reverse transcriptase inhibitors (NRTI), non-nucleoside reverse transcriptase inhibitors (NNRTI), purine nucleosides, chemokine receptor antagonists, interleukins, or combinations thereof. In certain embodiments, an NNRTI comprises: etravirine, efavirenz, nevirapine, rilpivirine, delavirdine, nevirapine, or combinations thereof. In certain embodiments an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate, or combinations thereof. In certain embodiments, a protease inhibitor comprises: amprenavir, tipranavir, indinavir, saquinavir mesylate, lopinavir, ritonavir, Fosamprenavir Calcium, darunavir, atazanavir sulfate, nelfinavir mesylate, or combinations thereof. In certain embodiments an integrase inhibitor comprises: raltegravir, dolutegravir, elvitegravir, or combinations thereof.

In certain embodiments, a method of preventing or treating lymphopenia in a subject suffering from an infection is provided that comprises administering to the subject a composition comprising a therapeutically effective amount of IL-15, an IL-15 superagonist, an IL-15 mutant, or combinations thereof. Preferably, the IL-15, IL-15 superagonist, IL-15 mutant, or combinations thereof, increase lymphocyte counts in peripheral tissues, lymphocyte activity, or a combination thereof. In certain embodiments, the IL-15 superagonist may be an IL-15:IL-15Rα complex. In certain embodiments, the method further comprises administering one or more chemotherapeutic agents, compounds, cytokine antagonists, cytokine receptor antagonists, cytokines, chemokines, growth factors, hpG-CSF, EPO, lymphopoietin, thrombopoietin, hormones, adoptive cell therapy, anti-viral agents, anti-microbial agents, anti-fungal agents, anti-parasitic agents, viral entry receptor antagonists, checkpoint inhibitors, adjuvants, or combinations thereof. In certain embodiments, the infection comprises a viral infection, bacterial infection, a parasitic infection, a fungal infection, or combinations thereof. Common infectious etiologies of lymphopenia include HIV infection, histoplasmosis, influenza infection, malaria, viral hepatitis, tuberculosis, typhoid fever, and sepsis.

In certain embodiments, a pharmaceutical composition comprises a therapeutically effective amount of IL-15, an IL-15 superagonist, an IL-15 mutant, or combinations thereof, and one or more chemotherapeutic agents, compounds, cytokine antagonists, cytokine receptor antagonists, cytokines, chemokines, growth factors, hpG-CSF, EPO, lymphopoietin, thrombopoietin, hormones, adoptive cell therapy, anti-viral agents, anti-microbial agents, anti-fungal agents, anti-parasitic agents, checkpoint inhibitors, adjuvants, or combinations thereof.

In certain embodiments, a pharmaceutical composition comprises a therapeutically effective amount of an IL-15:IL-15Rα complex and one or more chemotherapeutic agents, compounds, cytokine antagonists, cytokine receptor antagonists, cytokines, chemokines, growth factors, hpG-CSF, EPO, lymphopoietin, thrombopoietin, hormones, adoptive cell therapy, anti-viral agents, anti-microbial agents, anti-fungal agents, anti-parasitic agents, checkpoint inhibitors, adjuvants, or combinations thereof.

In certain embodiments, the IL-15:IL-15Rα complex is an IL-15N72D:IL-15RαSu/Fc complex (ALT-803, also known as nogapendekin alfa-inbakicept) comprising a dimeric IL-15RαSu/Fc and two IL-15N72D molecules.

Exemplary effective doses of the IL-15, an IL-15 superagonist, IL-15 mutants or combinations thereof, include between 0.1 μg/kg and 100 mg/kg body weight, e.g., 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, or 900 μg/kg body weight or 1, 2, 3, 4, 5, 6, 7, 8, 9, 10, 20, 30, 40, 50, 60, 70, 80, 90, or 100 mg/kg body weight.

In some cases, the IL-15, IL-15 superagonist, IL-15 mutant IL-15 mutants or combinations thereof, are administered daily, e.g., every 24 hours. In certain embodiments, the ALT-803 is administered continuously or several times per day, e.g., every 1 hour, every 2 hours, every 3 hours, every 4 hours, every 5 hours, every 6 hours, every 7 hours, every 8 hours, every 9 hours, every 10 hours, every 11 hours, or every 12 hours.

Exemplary effective daily doses of IL-15, an IL-15 superagonist, IL-15 mutant, or combinations thereof, include between 0.1 μg/kg and 100 μg/kg body weight, e.g., 0.1, 0.3, 0.5, 1, 5, 10, 15, 20, 25, 30, 35, 40, 45, 50, 55, 60, 65, 70, 75, 80, 85, 90, 95, or 99 μg/kg body weight.

Alternatively or additionally, the IL-15, IL-15 superagonist, IL-15 mutant, or combinations thereof, are administered about once per week, e.g., about once every 7 days. In certain embodiments, the ALT-803 is administered twice per week, three times per week, four times per week, five times per week, six times per week, or seven times per week. Exemplary effective weekly doses of ALT-803 include between 0.0001 mg/kg and 4 mg/kg body weight, e.g., 0.001, 0.003, 0.005, 0.01. 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, or 4 mg/kg body weight. For example, an effective weekly dose of IL-15, an IL-15 superagonist, IL-15 mutant, or combinations thereof, is between 0.1 μg/kg body weight and 400 μg/kg body weight. Alternatively or additionally, IL-15, an IL-15 superagonist, an IL-15 mutant, or combinations thereof, are administered at a fixed dose or based on body surface area (i.e., per m²).

In some cases, subjects receive two 6-week cycles consisting of 4 weekly intravenous doses of IL-15, an IL-15 superagonist, an IL-15 mutant, or combinations thereof, followed by a 2-week rest period. Ultimately, the attending physician decides the appropriate amount and dosage regimen.

Exemplary effective doses of the IL-15:IL-15Rα complex (ALT-803) include between 0.1 μg/kg and 100 mg/kg body weight, e.g., 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, or 900 μg/kg body weight or 1, 2, 3, 4, 5, 6, 7, 8, 9, 10, 20, 30, 40, 50, 60, 70, 80, 90, or 100 mg/kg body weight.

Exemplary effective daily doses of ALT-803 include between 0.1 μg/kg and 100 μg/kg body weight, e.g., 0.1, 0.3, 0.5, 1, 5, 10, 15, 20, 25, 30, 35, 40, 45, 50, 55, 60, 65, 70, 75, 80, 85, 90, 95, or 99 μg/kg body weight.

In some cases, subjects receive two 6-week cycles consisting of 4 weekly ALT-803 intravenous doses followed by a 2-week rest period. Ultimately, the attending physician or veterinarian decides the appropriate amount and dosage regimen.

In certain embodiments, the compositions described herein are administered systemically, intravenously, subcutaneously, intramuscularly, intraperitoneally, intravesically, or by instillation. In combination therapies, the agent of interest and ALT-803 may be administered simultaneously or sequentially.

In certain embodiments, preferred methods and combinations may comprises administration of a combination of two or more of IL-15, an IL-15 superagonist, an IL-15 mutant.

In certain other embodiments, methods and combinations may comprises administration of an IL-15 superagonist or an IL-15 mutant, but not IL-15. That is, in certain embodiments, method and compositions do not include use of IL-15.

Increases in lymphocyte counts in peripheral tissues, lymphocyte activity, or a combination thereof as referred to herein can be determined by established procedures including e.g. immunohistochemistry or flow cytometry. Peripheral tissue in which lymphocyte counts are assessed can include a subject's e.g. blood, skin, mucosa, or mucosa.

In other embodiments, pancytopenia treatment with IL-15, IL-15 superagonist, an IL-15 mutant, or combinations thereof, increases blood cell counts by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% compared to baseline control.

In other embodiments, lymphopenia treatment with IL-15, IL-15 superagonist, an IL-15 mutant, or combinations thereof, increases circulating immune cell numbers and/or activity by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% compared to baseline control.

In other embodiments, pancytopenia treatment with ALT-803 increases blood cell counts by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, compared to baseline control.

In other embodiments, lymphopenia treatment with ALT-803 increases circulating immune cell numbers and/or activity by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% compared to baseline control.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing, the preferred materials and methods are described herein. In describing and claiming the present invention, the following terminology will be used. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. For example, “an element” means one element or more than one element. Thus, “a cell,” for example, includes a plurality of cells of the same type. Furthermore, to the extent that “including,” “includes,” “having,” “has,” “with,” or variants thereof are used, such terms are intended to be inclusive in a manner similar to “comprising.”

“About” as used herein when referring to a measurable value such as an amount, a temporal duration, and the like, is meant to encompass variations that would round to the specified value at the next significant digit. Where particular values are described in the application and claims, unless otherwise stated the term “about” meaning within an acceptable error range for the particular value should be assumed.

“Antibody” and “immunoglobulin” convey both polyclonal and monoclonal antibodies. The preferred antibody is a monoclonal antibody reactive with the antigen. “Antibody” also encompasses mixtures of more than one antibody reactive with the antigen (e.g., a cocktail of different types of monoclonal antibodies reactive with the antigen). “Antibody” further encompasses whole antibodies, biologically functional fragments thereof, single-chain antibodies, and genetically altered antibodies such as chimeric antibodies comprising portions from more than one species, bifunctional antibodies, antibody conjugates, humanized, and human antibodies. Biologically functional antibody fragments, which can also be used, are antibody peptide fragments that are sufficient for binding to the antigen. Examples of useful antibody fragments include F(ab′)2, Fab′, Fab, and Fv capable of binding the epitope, antigen, or antigenic fragment of interest. A molecule “binds to” another molecule when one has a physicochemical affinity for the other.

“Anti-viral agent” refers to any molecule used for the treatment of a virus. “Anti-viral agent” include agents to alleviate symptoms associated with the virus (e.g., anti-pyretic agents, anti-inflammatory agents, chemotherapeutic agents, etc.). “Anti-viral agent” also includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNAs, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof. “Anti-viral agent” also refers to non-nucleoside reverse transcriptase inhibitors (NNRTIs), nucleoside reverse transcriptase inhibitors (NRTIs), analogs, variants, etc.

“Cancer” conveys a disease, condition, trait, genotype or phenotype characterized by unregulated cell growth or replication. “Cancer” includes colorectal cancer, leukemia (e.g., acute myelogenous leukemia (AML), chronic myelogenous leukemia (CML), acute lymphocytic leukemia (ALL), and chronic lymphocytic leukemia, AIDS related cancers such as Kaposi's sarcoma), breast cancers, bone cancers (e.g., osteosarcoma, chondrosarcomas, Ewing's sarcoma, fibrosarcomas, giant cell tumors, adamantinomas, and chordomas), brain cancers (e.g., meningiomas, glioblastomas, lower-grade astrocytomas, oligodendrocytomas, pituitary tumors, schwannomas, and metastatic brain cancers), cancers of the head & neck (e.g., various lymphomas such as mantle cell lymphoma, non-Hodgkin's lymphoma, adenoma, squamous cell carcinoma, laryngeal carcinoma, nasopharyngeal carcinoma), gallbladder & bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, lung cancer, bladder cancer, prostate cancer, lung cancer (including non-small cell lung carcinoma), pancreatic cancer, sarcomas, Wilms' tumor, cervical cancer, skin cancers, liposarcoma, epithelial carcinoma, renal cell carcinoma, gallbladder adeno carcinoma, parotid adenocarcinoma, and endometrial sarcoma. “Cancer” includes multidrug resistant cancers. “Cancer” also encompasses proliferative diseases and conditions, such as neovascularization associated with tumor angiogenesis, macular degeneration (e.g., wet/dry AMD), corneal neovascularization, diabetic retinopathy, neovascular glaucoma, myopic degeneration, restenosis, polycystic kidney disease.

“Cancer cell” conveys a cell exhibiting neoplastic phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell,” or “cancerous cell”, and encompasses cancer cells of a solid tumor, a semi-solid tumor, a primary tumor, a metastatic tumor, and the like.

“Cancer therapy” or “anti-cancer therapy” are used interchangeably to convey a therapy useful in treating cancer. Examples of anti-cancer therapeutic agents include, but are not limited to surgery, chemotherapeutic agents, immunotherapy, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN™), anti-CD20 antibodies, an epidermal growth factor receptor (EGFR) antagonist (e.g., a tyrosine kinase inhibitor), HER1/EGFR inhibitor (e.g., erlotinib (TARCEVA™)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also contemplated for use with the methods described herein.

A “chemotherapeutic agent” is a chemical compound useful in the treatment of cancer. Examples of chemotherapeutic agents include Erlotinib (TARCEVA™, Genentech/OSI Pharm.), Bortezomib (VELCADE™, Millennium Pharm.), Fulvestrant (FASLODEX™, Astrazeneca), Sutent (SU11248, Pfizer), Letrozole (FEMARA™, Novartis), Imatinib mesylate (GLEEVEC™, Novartis), PTK787/ZK 222584 (Novartis), Oxaliplatin (Eloxatin™, Sanofi), 5-FU (5-fluorouracil), Leucovorin, Rapamycin (Sirolimus, RAPAMUNE™, Wyeth), Lapatinib (GSK572016, GlaxoSmithKline), Lonafarnib (SCH 66336), Sorafenib (BAY43-9006, Bayer Labs.), and Gefitinib (IRESSA™, Astrazeneca), AG1478, AG1571 (SU 5271; Sugen), alkylating agents such as Thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozcicsin, carzcicsin and bizcicsin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin omega 1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, strcptonigrin, strcptozocin, tubcrcidin, ubenimcx, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacytidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK™ polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL™ paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ doxetaxel (Rhone-Poulenc Rorer, Antony, France); chloranbucil; GEMZAR™ gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in this definition of “chemotherapeutic agent” are: (i) anti-hormonal agents that regulate or inhibit hormone action on tumors such as anti-estrogens and selective estrogen receptor modulators (SERMs), including, for example, tamoxifen (including NOLVADEX™ (tamoxifen)), raloxifene, droloxifene, 4-hydroxytamoxifen, trioxifene, keoxifene, LY117018, onapristone, and FARESTON™ (toremifene); (ii) aromatase inhibitors that inhibit the enzyme aromatase, which regulates estrogen production in the adrenal glands, such as, for example, 4(5)-imidazoles, aminoglutethimide, MEGASE™ (megestrol acetate), AROMASIN™ (exemestane), formestanie, fadrozole, RIVISOR™ (vorozole), FEMARA™ (letrozole), and ARIMIDEX™ (anastrozole); (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in aberrant cell proliferation, such as, for example, PKC-alpha, Ralf and H-Ras; (viii) ribozymes such as a VEGF expression inhibitor (e.g., ANGIOZYME™ (ribozyme)) and a HER2 expression inhibitor; (ix) vaccines such as gene therapy vaccines, for example, ALLOVECTIN™ vaccine, LEUVECTIN™ vaccine, and VAXID™ vaccine; PROLEUKIN™ rIL-2; LURTOTECAN™ topoisomerase 1 inhibitor; ABARELIX™ rmRH; (x) anti-angiogenic agents such as bevacizumab (AVASTIN™, Genentech); and (xi) pharmaceutically acceptable salts, acids or derivatives of any of the above.

As used herein, “comprising,” “comprise,” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

A “growth inhibitory agent” conveys a compound or composition which inhibits growth of a cell either in vitro or in vivo. Examples include agents that significantly reduce the percentage of cells in S phase and agents that block cell cycle progression (at a place other than S phase)—such as agents that induce G1 arrest and M-phase arrest. Classical M-phase blockers include the vincas (e.g., vincristine and vinblastine), taxanes, and topoisomerase II inhibitors such as doxorubicin, epirubicin, daunorubicin, etoposide, and bleomycin. Agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. Taxanes (paclitaxel and docetaxel) are anticancer drugs both derived from the yew tree. Docetaxel (TAXOTERE™, Rhone-Poulenc Rorer), derived from the European yew, is a semisynthetic analog of paclitaxel (TAXOL™, Bristol-Myers Squibb). Paclitaxel and docetaxel promote the assembly of microtubules from tubulin dimers and stabilize microtubules by preventing depolymerization, which results in the inhibition of mitosis in cells.

“Effective amount” and “therapeutically effective amount” of a formulation or formulation component means a sufficient amount of the formulation or component, alone or in a combination, to provide the desired effect. For example, “an effective amount” means an amount of a compound, alone or in a combination, required to ameliorate the symptoms of a disease relative to an untreated patient. The effective amount of active compound(s) used for therapeutic treatment of a disease varies depending upon the manner of administration, the age, body weight, and general health of the subject. Ultimately, the attending physician or veterinarian will decide the appropriate amount and dosage regimen. Such amount is an “effective” amount.

As used herein, “IL-15:IL-15Rα fusion protein complex” is a complex having IL-15 non-covalently or covalently bound to IL-15Ra. IL-15Rα can be either soluble or membrane bound. In some embodiments, IL-15Rα is the soluble domain of the native IL-15Rα polypeptide. Soluble IL-15Rα can be the IL-15Rα sushi domain or IL-15RαΔE3. In some cases, soluble IL-15Rα is covalently linked to a biologically active polypeptide and/or to an IgG Fc domain. IL-15 can be either IL-15 or IL-15 covalently linked to a second biologically active polypeptide. In some cases, IL-15 is covalently bound to the IL-15Rα domain via a linker. IL-15 can also represent an IL-15 variant comprises one, two, three, four, or more amino acid variations relative to a reference sequence. In one embodiment the IL-15 is IL-15N72D. In one embodiment, the IL-15:IL-15Rα fusion protein complex is ALT-803.

“Interleukin-15” or “IL-15” refers to a polypeptide that has at least 85%, 90%, 95%, 96%, 97%, 98%, 99% or 100% sequence identity to a native mammalian IL-15 amino acid sequence, or a nucleotide encoding such a polypeptide. The human consensus IL-15 sequence is NWVNVISDLKKIEDLIQSMHIDATLYTESDVHPSCKVTAMKCFLLELQVISLESGDASIHDT VENLIILANNSLSSNGNVTESGCKECEELEEKNIKEFLQSFVHIVQMFINTS (SEQ ID NO:1). As used herein, “IL-15” is biologically active, meaning the polypeptide has functionality similar (75% or greater) to that of a native IL-15 protein in at least one functional assay. IL-15 cytokine regulates T cell and natural killer cell activation and proliferation. IL-15 and IL-2 share many biological activities, including binding to CD122, the IL-2β/IL-15β receptor subunit. CD8⁺ memory cell numbers are controlled by a balance between IL-15 and IL-2. IL-15 induces JAK kinase activation, as well as phosphorylation and activation of transcription activators STAT3, STATS, and STAT6. IL-15 increases expression of apoptosis inhibitor BCL2L1/BCL-x(L) to suppress apoptosis. IL-15 functional assays include T-cell proliferation (see, e.g., Montes et al. (2005) Clin Exp Immunol 142:292), and activation of NK cells, macrophages and neutrophils. Methods for isolating particular immune cell subpopulations and detecting proliferation (e.g., ³H-thymidine incorporation) are well known. Cell-mediated cellular cytotoxicity assays can measure NK cell, macrophage, and neutrophil activation. Cell-mediated cellular cytotoxicity assays, including release of isotopes (⁵¹Cr), dyes (e.g., tetrazolium, neutral red) or enzymes, have commercially available kits (Oxford Biomedical Research, Oxford, M; Cambrex, Walkersville, Md.; Invitrogen, Carlsbad, Calif.). IL-15 has also inhibits Fas mediated apoptosis (see, Demirci and Li, Cell Mol Immunol (2004) 1:123). Apoptosis assays, including for example, TUNEL assays and annexin V assays, also have commercially available kits (R&D Systems, Minneapolis, Minn.). See also, Coligan, et al., Current Methods in Immunology, 1991-2006, John Wiley & Sons.

“Native mammalian IL-15” or “wild type mammalian IL-15” refer to any naturally occurring interleukin-15 nucleic acid and amino acid sequences from a mammalian species. IL-15 nucleic acid and amino acid sequences are publicly available in gene databases, for example, GenBank through the National Center for Biotechnological Information. Exemplified native mammalian IL-15 nucleic acid or amino acid sequences can be from, for example, human, primate, canine, feline, porcine, equine, bovine, ovine, murine, etc. Accession numbers for exemplified native mammalian IL-15 nucleic acid sequences include NM_172174.2 (human preproprotein); NM_172175 (human); NM_000585.3 (human preproprotein); U19843 (macaque); DQ021912 (macaque); AB000555 (macaque); NM_214390 (porcine); DQ152967 (ovine); NM_174090 (bovine); NM_008357 (murine); NM_013129 (rattus); DQ083522 (water buffalo); XM_844053 (canine); DQ157452 (lagomorpha); and NM_001009207 (feline). Accession numbers for exemplified native mammalian IL-15 amino acid sequences include NP_000576.1 (human preproprotein); NP_751914 (human preproprotein); CAG46804 (human); CAG46777 (human); AAB60398 (macaque); AAY45895 (macaque); NP_999555 (porcine); NP_776515 (bovine); AAY83832 (water buffalo); ABB02300 (ovine); XP_849146 (canine); NP_001009207 (feline); NP_037261 (rattus); and NP_032383 (murine).

“Interleukin-15 receptor alpha” or “IL15Rα” refers to a polypeptide with at least 90%, 91%, 92%, 93%, 94%, 95%, 96%, 97%, 98% or 99% sequence identity to a native mammalian IL15Rα amino acid sequence, or a nucleotide encoding such a polypeptide, is biologically active, meaning the mutated protein (“mutein”) has functionality similar (75% or greater) to that of a native IL15Rα protein in at least one functional assay. IL15Rα is a cytokine receptor that specifically binds IL15 with high affinity. One functional assay is specific binding to a native IL-15 protein.

“Native mammalian interleukin-15 Receptor alpha (IL15Rα)” or “wild type il15Rα” refer to any naturally occurring IL-15 receptor alpha nucleic acid and amino acid sequences of the IL-15 receptor alpha from a mammalian species. IL-15 receptor alpha nucleic acid and amino acid sequences are publicly available in gene databases, for example, GenBank through the National Center for Biotechnological Information. Exemplified native mammalian IL-15Rα nucleic acid or amino acid sequences can be from, for example, human, primate, canine, feline, porcine, equine, bovine, ovine, murine, etc. Accession numbers for exemplified native mammalian IL-15Rα nucleic acid sequences include NM_172200.1 (human isoform 2); and NM_002189.2 (human isoform 1 precursor). Accession numbers for exemplified native mammalian IL-15Rα amino acid sequences include NP_751950.1 (human isoform 2); and NP_002180.1 (human isoform 1 precursor).

An “an IL-15 mutant” polypeptide as referred to herein includes polypeptides that differ from a sequence of a naturally occurring IL-15 polypeptides by multiple residues and preferably have a sequence identity of 95% or less (such as 94%, 93%, 92%, 91%, 90%, 88%, 85%, 82%, 80%, 78%, 75% or less sequence identity) relative to a native mammalian IL-15 amino acid sequence, or a nucleotide encoding such a native polypeptide. Preferred IL-15 mutants may have sequence differences from a naturally occurring IL-15 in two or more regions. For instance, an IL-15 mutant may suitably lack one or more of the first 48 amino acid residues (i.e., the N-terminal residues) of the IL-15 precursor protein and, second, include a substitution mutation of one or both of the glutamine (Q) residues in the C-terminal half of the polypeptide. Deletions may be of 1, 2, 3, 5, 10, 12, 15, 20, 25, 30, 35, 40, 45, or 48 of the 48 most N-terminal residues of the precursor protein. The deletion suitably can begin at the first residue and may or may not include contiguous amino acid residues. Substitution mutations include replacing each glutamine (N) with one or more (e.g., 1, 2, 3, 5, 10, or more) amino acids. For example, glutamines can be replaced with a single amino acid (i.e., the substitution mutation can be a point mutation), which may be aspartic acid (D) or any other natural or non-natural amino acid. The mature IL-15 that is mutated can be native mammalian IL-15 or wild type mammalian IL-15 as discussed above, and suitably may be human. A mutant IL-15 suitably can be joined to one or more heterologous polypeptides, which may constitute at least part of a naturally occurring protein. The non-IL-15 portion of the chimera may increase the circulating half-life of the mutant IL-15, serve as a label or tag (e.g., an antigenic tag or epitope tag), or confer some other desirable quality on the mutant IL-15. As used herein, “IL-15 mutant” does not encompass an IL-15 superagonist.

As used herein, “immune cells” include white blood cells (leukocytes), lymphocytes (T cells, B cells, natural killer (NK) cells), and myeloid-derived cells (neutrophil, eosinophil, basophil, monocyte, macrophage, dendritic cells).

“Immune effector cell,” as used herein, refers to a cell involved in an immune response, e.g., in the promotion of an immune effector response. Examples of immune effector cells include T cells, e.g., alpha/beta T cells and gamma/delta T cells, B cells, natural killer (NK) cells, natural killer T (NK-T) cells, mast cells, and myeloid-derived phagocytes. “Immune effector function” or “immune effector response,” as used herein, refer to functions or responses of an immune effector cell that enhances or promotes an immune attack of a target cell. For example, an immune effector function or response refers a property of a T or NK cell that promotes killing or the inhibition of growth or proliferation, of a target cell. In the case of a T cell, primary stimulation and co-stimulation are examples of immune effector function or response.

“In combination” in the context of the administration of a therapy to a subject refers to the use of more than one therapy for therapeutic benefit. “In combination” in the context of administration can also refer to the prophylactic use of a therapy to a subject when used with at least one additional therapy. The use of “in combination” does not restrict the order in which the therapies (e.g., a first and second therapy) are administered. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to cancer. The therapies are administered to a subject in a sequence and within a time interval such that the therapies can act together. In a particular embodiment, the therapies are administered to a subject in a sequence and within a time interval such that they provide an increased benefit relative to administration otherwise. Any additional therapy can be administered in any order with the other additional therapy.

“Neoplasia” conveys a disease or disorder characterized by excess proliferation or reduced apoptosis. Neoplastic conditions include, but are not limited to, cancers, sarcomas, tumors, leukemias, lymphomas, and the like. A neoplastic condition refers to the disease state associated with the neoplasia. Colon cancer (e.g., colorectal cancer), lung cancer and ovarian cancer are non-limiting examples of neoplastic conditions. Illustrative neoplasms for which the invention can be used include, but are not limited to leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). In particular embodiments, the neoplasia is multiple myeloma, beta-cell lymphoma, urothelial/bladder carcinoma or melanoma.

As used herein, “obtaining” as in “obtaining an agent” includes synthesizing, purchasing, or otherwise acquiring the agent.

“Optional” or “optionally” mean that the subsequently described event or circumstance can or cannot occur, and that the description includes instances where the event or circumstance occurs and instances where it does not.

As used herein, “or” is employed in its sense including “and/or” unless the content clearly dictates otherwise.

“Parenteral” administration of an immunogenic composition includes, e.g., subcutaneous (s.c.), intravenous (i.v.), intramuscular (i.m.), or intrasternal injection, or infusion techniques.

“Patient” or “individual” or “subject” are used interchangeably herein, and refer to a mammalian subject to be treated, with human patients being preferred. In some cases, the methods disclosed herein are used in experimental animals, in veterinary application, and in the development of animal models for disease, including, but not limited to, rodents including mice, rats, and hamsters, and primates.

As used herein, a “pharmaceutically acceptable” component/carrier etc. is suitable for use with humans and/or animals without undue adverse side effects (such as toxicity, irritation, and allergic response) commensurate with a reasonable benefit/risk ratio.

“Preventing” and “prevention” refer to administration of an agent or composition to a clinically asymptomatic individual who is susceptible or predisposed to a particular adverse condition, disorder, or disease, to prevent occurrence of symptoms and/or their underlying cause.

By “reduces” is meant a negative alteration of at least 5%, 10%, 25%, 50%, 75%, or 100%.

“Soluble IL-15Rα” or “sIL-15Rα” refers to forms of IL-15Rα lacking transmembrane anchor portions of the receptor. sIL-15Rα can be secreted without being anchored to the plasma membrane. Exemplary sIL-15α include aa31-205 and aa31-185 of the native IL-15Rα.

“Treating” and “treatment” refer to administering an agent or formulation to a clinically symptomatic individual afflicted with an adverse condition, disorder, or disease, to reduce severity and/or frequency of symptoms, eliminate the symptoms and/or their underlying cause, and/or facilitate improvement or remediation of damage. Although not precluded, treating a disorder or condition does not require that the disorder, condition or symptoms associated therewith be completely eliminated.

Treatment of cancer patients may include any of the following: adjuvant therapy (also called adjunct therapy or adjunctive therapy) to destroy residual tumor cells that may be present after the known tumor is removed by the initial therapy (e.g. surgery), thereby preventing possible cancer reoccurrence; neoadjuvant therapy given prior to the surgical procedure to shrink the cancer; induction therapy to cause a remission, typically for acute leukemia; consolidation therapy (also called intensification therapy) given once a remission is achieved to sustain the remission; maintenance therapy given in lower or less frequent doses to assist in prolonging a remission; first line therapy (also called standard therapy); second (or 3rd, 4th, etc.) line therapy (also called salvage therapy) is given if a disease has not responded or reoccurred after first line therapy.

As used herein, “tumor” means a mass of transformed cells engaged in neoplastic uncontrolled cell multiplication and, at least in part, containing angiogenic vasculature. Abnormal neoplastic cell growth is rapid and continues even after the stimuli that initiated the new growth have ceased. “Tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e. a metastatic tumor), a tumor also can be nonmalignant (i.e. non-metastatic tumor).

Certain of the methods disclosed herein include a step that involves comparing a value, level, feature, characteristic, property, etc. to a “suitable control”, referred to interchangeably herein as an “appropriate control”. A “suitable control” or “appropriate control” is a control or standard familiar to one of ordinary skill in the art useful for comparison purposes. In one embodiment, a “suitable control” or “appropriate control” is a value, level, feature, characteristic, property, etc. determined prior to performing a treatment and/or agent administration methodology, as described herein. For example, a transcription rate, mRNA level, translation rate, protein level, biological activity, cellular characteristic or property, genotype, phenotype, etc. can be determined prior to introducing a treatment. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

Genes: All genes, gene names, and gene products disclosed herein correspond to homologs from any species for which the compositions and methods disclosed herein are applicable. When a gene or gene product from a particular species is disclosed, this disclosure is intended to be exemplary only, and is not to be interpreted as a limitation unless the context in which it appears clearly indicates. Thus, for example, for the genes or gene products disclosed herein, encompass homologous and/or orthologous genes and gene products from other species. Genbank and NCBI submissions indicated by accession number cited herein are incorporated herein by reference.

Ranges: throughout this disclosure, various ranges are mentioned. Description in range format is merely for convenience and brevity and should not be construed as an inflexible limitation. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic representation of the clinical study protocol.

FIG. 2 is a schematic representation of the subjects' disposition.

FIG. 3 is a graph demonstrating Mean Concentration—Time Profile following administration of 10 μg/kg ALT-803 (PK Population).

FIG. 4 is a graph demonstrating Mean Concentration—Time Profile following administration of 20 μg/kg ALT-803 (PK Population).

FIG. 5 is a graph demonstrating the induction of immune cell proliferation following ALT-803 administration.

FIG. 6 is a graph demonstrating mean serum levels of IL-6 following ALT-803 administration.

FIG. 7 is a graph demonstrating mean serum levels of IL-10 following ALT-803 administration.

FIG. 8 is a graph demonstrating mean serum levels of IFN-γ following ALT-803 administration.

FIG. 9 is a graph demonstrating lymphocyte count over time. BL=baseline; D=day.

FIG. 10 is a graph demonstrating pulse rate over time. BL=baseline; D=day.

FIG. 11 is a graph demonstrating temperature over time. BL=baseline; D=day.

FIG. 12 is a table of the schedule of assessments for participants of Study Period 1 and Study Period 2.

### DETAILED DESCRIPTION

Pancytopenia is the simultaneous presence of anemia, lymphopenia, and thrombocytopenia. In subjects presenting with pancytopenia, hemoglobin (Hb) is less than 13.5 g/dl in males or 11.5 g/dl in females. Pancytopenic leucocyte counts are <4×10³/L and platelets counts <150×10³/L. Initially, mild impairment in marrow function may go undetected. Pancytopenia may become apparent only during times of stress or increased demand (e.g., bleeding or infection). Varieties of hematopoietic and non-hematopoietic conditions manifest with features of pancytopenia. The underlying mechanisms are: decrease in hematopoietic cell production, marrow replacement by abnormal cells, suppression of marrow growth and differentiation, ineffective hematopoiesis with cell death, defectively formed cells removed from the circulation, antibody mediated sequestration or destruction of cells and trapping of cells in a hypertrophied and over active reticuloendothelial system.

Lymphopenia is a total lymphocyte count of <1000/μL in adults or <3000/μL in children under 2 years old. Sequelae include opportunistic infections and an increased risk of malignant and autoimmune disorders. If the complete blood count (CBC) reveals lymphopenia, testing for immunodeficiency and analysis of lymphocyte subpopulations should follow. Treatment is directed at the underlying disorder. Normal lymphocyte counts in adults are 1000 to 4800/μL; in children <2 yr, 3000 to 9500/μL. At age 6 yr, the lower limit of normal is 1500/μL.

IL-15, IL-15 Mutants, IL-15 Soluble Fusion Protein Complex Compositions.

The compositions embodied herein comprise one or more IL-15 soluble fusion protein complexes, IL-15, IL-15 superagonist, IL-15 mutant, or combinations thereof. In certain embodiments, the soluble fusion protein complexes include an IL-15 polypeptide, IL-15 variant, or a functional fragment thereof and a soluble IL-15Rα polypeptide or a functional fragment thereof. In some cases, one or both of the IL-15 and IL-15Rα polypeptides further include an immunoglobulin Fc domain or a functional fragment thereof. For example, the IL-15/IL-15Rα complex is an IL-15N72D:IL-15RαSu/Fc complex (ALT-803), wherein the ALT-803 comprises dimeric IL-15RαSu/Fc and two IL-15N72D molecules. IL-15 is a potent cytokine that increases CD8+T and NK cell numbers and function in experimental models.

Interleukin-15 (IL-15) and IL-15 Mutants.

IL-15 is a four-α-helix protein belonging to a cytokine family consisting of IL-2, IL-4, IL-7, IL-9, & IL-21. IL-15 signals through a receptor complex composed of the IL-2/IL-15 receptor β (IL-15Rβ) (CD122) subunit, which is shared with IL-2 and the common gamma chain (γC) (CD132) receptor subunit, which is also utilized by all of the additional family members. While IL-15Rα does not have a crucial direct role in IL-15 signaling per se, it is a critical component of the IL-15 cytokine-receptor complex. IL-15Rα is a transmembrane protein with very high affinity for IL-15 that facilitates IL-15 trafficking from the endoplasmic reticulum (ER) through the cytoplasm and presentation of IL-15/IL-15Rα complexes on the cell surface. In addition to remaining associated throughout cytoplasmic and cell surface expression, IL-15/IL-15Rα can also be cleaved as a complex into the extracellular space. These peculiarities of IL-15 and IL-15R subunits facilitate unique mechanisms of cytokine delivery.

IL-2/15Rβ and γC expression is believed to be the major attribute conferring IL-15 responsiveness. These receptors are present on many hematopoietic cells; however, the IL-2/15Rβ expression is highest on CD8 T cells and NK cells. Upon ligand binding, the IL-2/15Rβ and γC subunits stimulate Janus kinase (Jak)1, Jak3, and signal transducer and activator of transcription (STAT)-5 pathway. After phosphorylation, STATS homo-dimerizes, translocates to the nucleus, and promotes the transcription of target genes. IL-15 stimulates PI3K-AKT and RAS-MAPK pathways (Ali & al. (2015) Front Immunol. 6:355). Altogether, IL-15 signaling stimulates several pathways leading to increased cellular growth, decreased apoptosis, and enhanced immune cell activation & migration. At a low static level, these responses have a crucial role in development, function, and survival of CD8 T cells, NK cells, NKT cells and intestinal intraepithelial lymphocytes. This is reflected by the major deficiency of these populations in both IL-15^{-/-} and IL-15Rα^{-/-} mice (Kennedy et al. (2000) J Exp Med. 191(5):771-80; Lodolce et al. (1998) Immunity 9(5):669-76). Phenotypic similarity between these knockout strains shows the reliance of IL-15Rα for IL-15-mediated effects. Endogenously-expressed IL-15 mediates lymphocyte homeostasis. IL-15 delivered exogenously at supra-physiological levels induces the selective activation and proliferation in CD8 T cells and NK cells—the cell types most amenable to mediating anti-tumor response. IL-15 cell specificity is one attribute that makes it so attractive for immunotherapy.

IL-15 and IL-15Rα are widely expressed by most cell types, including both hematopoietic and non-hematopoietic cells. Expression is highest among myeloid cells. IL-15/IL-15Rα complexes can stimulate neighboring or opposing cells in trans through the IL-15Rβ/γC. This stimulation requires cell-cell contact and is referred to as transpresentation. Under steady-state conditions, trans-presentation is the primary mode of action for IL-15-mediated development and homeostasis of CD8 T cells, NK cells, NKT cells, and intraepithelial lymphocytes (Mortier et al. (2009) Immunity 31(5):811-22). Trans-presentation offers a tighter regulation than that of a secreted cytokine. Nonetheless, soluble (s) IL-15/IL-15Rα complexes are also cleaved from the cell surface in response to inflammatory signals, such as TLR ligation, type I Interferons, and CD40 ligation (Anthony et al. (2015) PLoS One 10(3):e0120274). Production of sIL-15 complexes is transient and provides a short-lived, but strong burst of IL-15 activity. IL-15Rα binding of IL-15 is not only a platform for IL-15 delivery but also increases IL-15 half-life and affinity for IL-15Rβ/γC. Indeed, sIL-15 complexes stimulate IL-15 responses more powerfully than unassociated rIL-15 (Rubinstein et al. (2006) Proc Natl Acad Sci USA 103(24):9166-71).

Recombinant interleukin-15 (rIL-15) is limited as a treatment by IL-15Rα availability. IL-15Rα stabilizes IL-15 and increases its biological activity. Because unassociated IL-15 is not found naturally in vivo, IL-15 bound to IL-15Rα resembles the physiological form of IL-15 and has a higher affinity than free IL-15 for IL-15Rβ/γC (Mortier et al. (2006) J Biol Chem. 281(3):1612-19). Soluble IL-15/IL-15Rα complexes are advantageously not dependent on trans-presentation or cell/cell interaction. Overall, in multiple models systems, regardless of formation, sIL-15/IL-15Rα complexes are significantly more potent than native IL-15 both in vitro and in vivo (Watson et al. (2016) Biomaterials 105:195-205). Furthermore, these IL-15 agonists can also stimulate tumor-specific lymphocytes (TILs) and NK cells ex vivo (Desbois et al. (2016) J Immunol. 197(1):168-78).

One strategy for generating sIL-15 complexes combines rIL-15 with recombinant soluble murine IL-15Rα (sIL-15Rα) linked to the Fc portion of the human IgG1 antibody (IL-15/IL-15Rα-Fc complex). The fusion of the IgG Fc domain enhances many proteins' plasma half-life (Czajkowsky et al. (2012) EMBO Mol Med. 4(10):1015-28). A second strategy to enhance IL-15 potency and to simplify production uses a fusion protein, consisting of the NH2-terminal (amino acids 1-77, sushi+) cytokine-binding domain of human IL-15Rα coupled to human IL-15 via a 20-amino acid flexible linker. This fusion protein, referred to as protein receptor-linker-IL-15 (RLI) acts as an IL-15 superagonist with an increased serum half-life and biological activity similar to complexed IL-15/IL-15Rα-Fc (Mortier et al. (2006) J Biol Chem. 281(3):1612-19). Other IL-15 based molecules include P22339 (Hu et al. (2018) Scientific Reports 8:7675).

IL-15 Superagonist:

ALT-803 comprises an IL-15 mutant with increased binding to IL-2Rβγ and enhanced biological activity (U.S. Pat. No. 8,507,222, incorporated herein by reference). ALT-803 is also known in the literature under the names N-803 and nogapendekin-alfa-inbakicept. This super-agonist IL-15 mutant was described in Zhu et al. (2009) J Immunol 183:3598. ALT-803 is also described in several co-pending applications (e.g., U.S. Ser. No. 12/151,980 & 13/238,925). ALT-803 in combination with a soluble IL-15α receptor fusion protein (IL-15RαSu/Fc) results in a protein complex with highly potent IL-15 activity in vitro and in vivo (Han et al. (2011) Cytokine, 56:804-10). IL-15:IL-15Rα fusion protein complexes can have IL-15 non-covalently bound to the soluble IL-15Rα domain. In some cases, the soluble IL-15Rα is covalently linked to a biologically active polypeptide and/or to an IgG Fc domain. The IL-15 can be either IL-15 or IL-15 covalently linked to a second biologically active polypeptide. The crystal structure of the IL-15:IL-15Rα complex is shown in Chirifu et al. (2007) Nat Immunol 8:1001-07.

ALT-803 has a half-life of 25 hours following i.v. administration in mice. ALT-803 biodistributes to lymphoid organs and shows impressive anti-tumor activity against aggressive solid and hematological tumor models in immunocompetent mice. ALT-803 can be administered as a monotherapy using a twice weekly or weekly i.v. dose regimen, or as combination therapy with an antibody or immunogen (e.g., Bacillus Calmette-Guérin).

Blood Cellular Components:

Hematopoiesis is the formation of blood cellular components. All cellular blood components derive from hematopoietic stem cells (HSCs). Healthy human adults produce approximately 10¹¹-10¹² new blood cells daily to maintain steady state levels in the peripheral circulation. Several blood cells types circulate: red blood cells, white blood cells, and platelets. Five differentiation pathways from the HSCs produce these various cell types: erythropoiesis; lymphopoiesis; granulopoiesis; monopoiesis; and thrombopoiesis.

Red blood cells, also called erythrocytes, carry oxygen. Quantifying reticulocytes—immature red blood cells—gives an estimate of erythropoietic rate.

Lymphocytes power the adaptive immune system. They derive from common lymphoid progenitors. The lymphoid lineage consists of T-cells, B-cells, & natural killer cells.

Myeloid cells include granulocytes, megakaryocytes, and macrophages. Myeloid cells are involved in innate immunity and blood clotting. Granulopoiesis (or granulocytopoiesis) is hematopoiesis of granulocytes, except of mast cells which are granulocytes but with an extramedullar maturation. Megakaryocytopoiesis is hematopoiesis of megakaryocytes.

Each blood cell type can be identified by cell-specific markers and by routine assays, e.g. ELISA, FACS analysis etc.

Lymphocytes:

CD8⁺ cytotoxic lymphocytes and NK cells primarily mediate anti-viral and anti-neoplastic immune responses. Numerous cytokines contribute to cytotoxic immunity, including IL-11 and the interferons. At least two cytokines contribute to B-lymphocyte maturation in the bone marrow: IL-7; and IL-11. IL-7 is critically important to both B and T cell development through its production by stromal tissue of the bone marrow and thymus. IL-7 interacts with lymphoid precursors. In addition, IL-7 stimulates cytotoxic T & NK proliferation and differentiation and stimulates monocytes & macrophages tumoricidal activity. The central importance of IL-7 to lymphoid maturation is reflected in severe combined immune deficiency resulting from the absence of either IL-7 or functional IL-7 receptors (IL-7 receptor α [CD127] or common γ chain).

Subclasses of T helper (T_{H}) lymphocytes can be identified based on cytokines. Naive T_{H}0 cells produce primarily IL-2. Human T_{H}1 cells primarily produce IFN-γ and TNF-β but not IL-4 & IL-5. T_{H}2 cells more prominently produce IL-4, IL-5, IL-9, & IL-13, but not IFN-γ. T_{H}1 lymphocytes promote cell-mediated immune responses. T_{H}1 lymphocytes are important in antibody-dependent immunity. T_{H}17 cells are more important in the T cell-mediated response to extracellular pathogens, and likely contribute to autoimmune diseases. T_{H}2 lymphocytes produce IL-4, IL-5, & IL-13. Veldhoen et al. (2008) Nat Immunol 9:1341-46 describe a subclass of T_{H}2 cells characterized by prominent IL-9 production (TH9 cells).

Accordingly, in certain embodiments, a method of preventing or treating pancytopenia in a subject, comprises administering to the subject a composition comprising a therapeutically effective amount of IL-15, an IL-15 superagonist, an IL-15 mutant, or combinations thereof, wherein the IL-15, IL-15 superagonist, IL-15 mutant, or combinations thereof, increase the number of blood cells in the subject. In certain embodiments, a method of preventing or treating pancytopenia in a subject, comprises administering to the subject a composition comprising a therapeutically effective amount of an IL-15:IL-15Rα complex, wherein the IL-15:IL-15Rα complex increases the number of blood cells in the subject.

In certain embodiments, the pancytopenia is induced by one or more factors comprising: anemia, cancer, infectious agents, immune disorders, genetic disorders, bone marrow disorders, hypersplenism, organ injury, kidney disease, transplantations, myelodysplastic syndromes, gastrointestinal disorders, chronic virus infection, recurrent virus infection, age related, drug-induced, chemotherapy, surgery, radiation therapy, steroid therapy, splenic sequestration, toxins, chemical exposure, radiation or combinations thereof. In certain embodiments the pancytopenia is lymphopenia.

In certain embodiments, lymphopenia is treated following radiation therapy. In certain embodiments, the radiation therapy comprises administering less than 35 Gray of radiation. In certain embodiments, the radiation therapy comprises administering less than 40 Gray of radiation. In certain embodiments, the radiation therapy comprises administering less than 45 Gray of radiation. In certain embodiments, the radiation therapy comprises administering less than 50 Gray of radiation. In certain embodiments, the radiation therapy comprises administering less than 35 Gray of radiation over 5 weeks. In certain embodiments, the radiation therapy comprises administering less than 40 Gray of radiation over 5 weeks. In certain embodiments, the radiation therapy comprises administering less than 45 Gray of radiation over 5 weeks. In certain embodiments, the radiation therapy comprises administering less than 50 Gray of radiation over 5 weeks. In certain embodiments, the lymphopenia treatment is administered on the same day as administration of radiation therapy. In certain embodiments, the lymphopenia treatment is administered within 24 hours of administration of radiation therapy. In certain embodiments, the lymphopenia treatment is administered within 48 hours of administration of radiation therapy.

In certain embodiments, a method of preventing or reducing lymphopenia in a subject in need thereof, comprises administering to the subject a composition comprising a therapeutically effective amount of IL-15, an IL-15 superagonist, an IL-15 mutant, or combinations thereof, wherein the IL-15, IL-15 superagonist, IL-15 mutant, or combinations thereof, increase lymphocyte counts in peripheral tissues, lymphocyte activity, or a combination thereof.

In certain embodiments, a method of preventing or treating lymphopenia in a subject in need thereof, comprises administering to the subject a composition comprising a therapeutically effective amount of an IL-15:IL-15Rα complex, wherein the IL-15:IL-15Rα complex increases lymphocyte counts in peripheral tissues, lymphocyte activity, or a combination thereof.

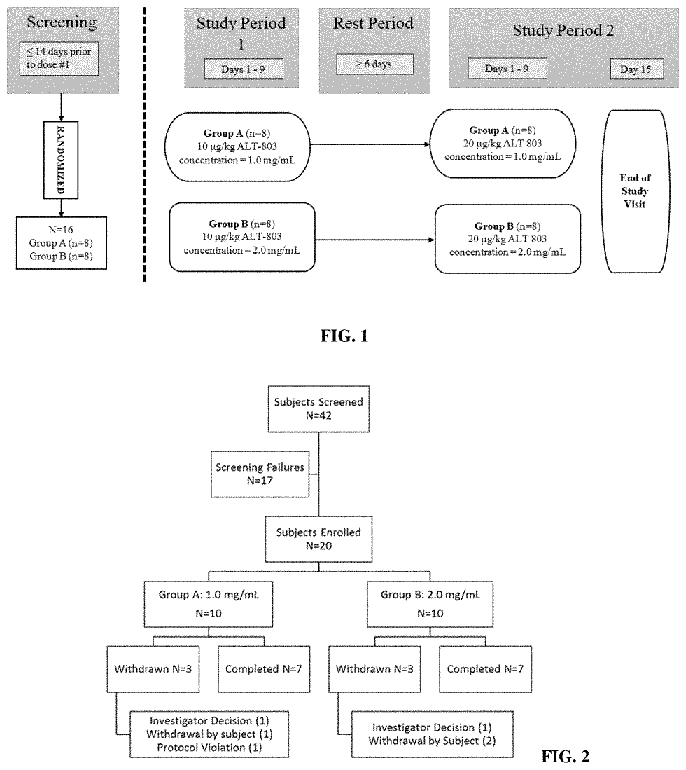

In other embodiments, treatment of pancytopenia with IL-15, an IL-15 superagonist, an IL-15 mutant, or combinations thereof increases the number of blood cells by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% as compared to a baseline control. The complete blood cell count can be determined by routine assays, which quantify the amount and type of blood cell. Samples from subjects can be taken at various intervals. Cell counts can be quantified and identified as per detailed protocols in Example 1 and FIGS. 1 & 5-9.

In other embodiments, treatment of lymphopenia with IL-15, an IL-15 superagonist, an IL-15 mutant, or combinations thereof increases circulating immune cell numbers and/or activity by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% as compared to a baseline control. The white blood cell count can be determined by routine assays which quantify the amount and type of lymphocyte. Samples can be taken at various intervals. Cell counts can be quantified and identified as per detailed protocols in Example 1.

In other embodiments, ALT-803 treatment of pancytopenia increases the number of blood cells by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% as compared to a baseline control. The complete blood cell count can be determined by routine assays which quantify the amount and type of blood cell. Samples from subjects can be taken at various intervals. Cell counts can be quantified and identified as per detailed protocols in Example 1 and FIGS. 1 & 5-9.

In other embodiments, ALT-803 treatment of lymphopenia increases circulating immune cell numbers and/or activity by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% as compared to a baseline control. The white blood cell count can be determined by routine assays to quantify the amount and type of lymphocyte. Samples can be taken at various intervals. Cell counts can be quantified and identified as per the detailed protocols in Example 1.

In certain embodiments, the method of preventing or treating pancytopenia or lymphopenia further comprises administering one or more chemotherapeutic agents, compounds, cytokine antagonists, cytokine receptor antagonists, cytokines, chemokines, growth factors, hpG-CSF, EPO, lymphopoietin, thrombopoietin, hormones, adoptive cell therapy, anti-viral agents, viral entry receptor antagonists, checkpoint inhibitors, adjuvants, or combinations thereof.

In certain embodiments, the cytokines are T_{H}1-inducing cytokines. In certain embodiments, the T_{H}1-inducing cytokines comprise IL-2, IL-10, IL-12, IL-18, IL-27, tumor necrosis factor-alpha (TNFα), tumor necrosis factor-beta (TNFβ), interferons, or combinations thereof.

In certain embodiments the cytokines are T_{H}2-inducing cytokines. In certain embodiments the T_{H}2-inducing cytokines comprise IL-4, IL-5, IL-6, IL-9, IL-13, IL-19, IL-25, IL-31, IL-33, interferons, or combinations thereof.

In certain embodiments, the adoptive cell therapy comprises administration of lymphocytes, hematopoietic stem cells or combinations thereof.

In certain embodiments, the subject is suffering from a disease or disorder associate with lymphopenia. In certain embodiments, the disease or disorder associate with lymphopenia comprises: cancer, infectious agents, immune disorders, genetic disorders, bone marrow disorders, hypersplenism, organ injury, kidney disease, transplantations, myelodysplastic syndromes, gastrointestinal disorders, chronic virus infection, recurrent virus infection, age related lymphopenia or combinations thereof. Genetic disorders comprise: Wiskott-Aldrich syndrome, adenosine deaminase deficiency, severe combined immunodeficiency disorder, purine nucleoside phosphorylase deficiency, ataxia-telangiectasia or DiGeorge anomaly.

Infection:

The compositions embodied herein are applicable to treating pancytopenia or lymphopenia resulting from any cause. In certain embodiments, the lymphopenia is caused by an infection. The human immunodeficiency virus (HIV) is used herein merely for illustrative purposes and is not meant to be limiting.

In certain embodiments, a method of preventing or treating lymphopenia in a subject infected with a pathogen (e.g., HIV), comprises administering to the subject a composition comprising a therapeutically effective amount of an IL-15:IL-15Rα complex, wherein the IL-15:IL-15Rα complex increases lymphocyte counts in peripheral tissues, lymphocyte activity, or a combination thereof. In certain embodiments, the method further comprises administering one or more chemotherapeutic agents, compounds, cytokine antagonists, cytokine receptor antagonists, cytokines, chemokines, chemokine receptor antagonists, viral entry receptor antagonists, growth factors, hpG-CSF, EPO, lymphopoietin, adoptive cell therapy, anti-viral agents, checkpoint inhibitors, adjuvants, or combinations thereof.

Receptors for viral entry include the CD4 receptor to which the HIV gp120 attaches. The CD4 receptor is found on CD4 T-cells and macrophages. In certain embodiments, the antagonists inhibit binding of HIV to one or more receptors wherein the receptors comprise CD4, CXCR4, CXCR5, variants or combinations thereof.

Combination Therapies:

The compositions comprising the IL-15:IL-15Rα complex, IL-15, an IL-15 superagonist, IL-15 mutant, or combinations thereof, wherein the IL-15, IL-15 superagonist, IL-15 mutant, or combinations thereof, can be included in the composition or administered as a second therapeutic agent or treatment with a second therapy (e.g., a therapeutic agent or therapy that is standard in the art).

Anti-Cancer Therapeutic Agents:

Exemplary therapeutic agents for use in combination therapy include chemotherapeutic agents. A “chemotherapeutic agent” is a chemical compound useful in the treatment of cancer. Examplary chemotherapeutic agents include Erlotinib (TARCEVA™, Genentech/OSI Pharm.), Bortezomib (VELCADE™, Millennium Pharm.), Fulvestrant (FASLODEX™, Astrazeneca), Sutent (SU11248, Pfizer), Letrozole (FEMARA™, Novartis), Imatinib mesylate (GLEEVEC™, Novartis), PTK787/ZK 222584 (Novartis), Oxaliplatin (Eloxatin™, Sanofi), 5-FU (5-fluorouracil), Leucovorin, Rapamycin (Sirolimus, RAPAMUNE™, Wyeth), Lapatinib (GSK572016, GlaxoSmithKline), Lonafarnib (SCH 66336), Sorafenib (BAY43-9006, Bayer Labs.), and Gefitinib (IRESSA™, Astrazeneca), AG1478, AG1571 (SU 5271; Sugen), alkylating agents such as Thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozcicsin, carzcicsin and bizcicsin synthetic analogs); cryptophycins (particularly cryptophycin 1 and cryptophycin 8); dolastatin; duocarmycin (including the synthetic analogs, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin omega 1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, strcptonigrin, strcptozocin, tubcrcidin, ubenimcx, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacytidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK™ polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL™ paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ doxetaxel (Rhone-Poulenc Rorer, Antony, France); chloranbucil; GEMZAR™ gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in this definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX™ (tamoxifen)), raloxifene, droloxifene, 4-hydroxytamoxifen, trioxifene, keoxifene, LY117018, onapristone, and FARESTON™ (toremifene); (ii) aromatase inhibitors that inhibit the enzyme aromatase, which regulates estrogen production in the adrenal glands, such as, for example, 4(5)-imidazoles, aminoglutethimide, MEGASE™ (megestrol acetate), AROMASIN™ (exemestane), formestanie, fadrozole, RIVISOR™ (vorozole), FEMARA™ (letrozole), and ARIMIDEX™ (anastrozole); (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in aberrant cell proliferation, such as, for example, PKC-alpha, Ralf and H-Ras; (viii) ribozymes such as a VEGF expression inhibitor (e.g., ANGIOZYME™ (ribozyme)) and a HER2 expression inhibitor; (ix) vaccines such as gene therapy vaccines, for example, ALLOVECTIN™ vaccine, LEUVECTIN™ vaccine, and VAXID™ vaccine; PROLEUKIN™ rIL-2; LURTOTECAN™ topoisomerase 1 inhibitor; ABARELIX™ rmRH; (x) anti-angiogenic agents such as bevacizumab (AVASTIN™, Genentech); and (xi) pharmaceutically acceptable salts, acids or derivatives of any of the above.

Checkpoint Inhibitors: The compositions embodied herein, can also include one or more checkpoint inhibitors. In one embodiment, the subject can be administered an agent which enhances the activity of an immune effector cell. For example, in one embodiment, the agent can inhibit a molecule that modulates or regulates (e.g., inhibits) immune response of an immune effector cell, e.g., T cell function. In some embodiments, the molecule that modulates or regulates immune response of an immune effector cell is an inhibitory molecule, also known as a checkpoint inhibitor. Inhibitory molecules, also referred to herein as checkpoint inhibitors, e.g., Programmed Death 1 (PD-1), can, in some embodiments, decrease the ability of an immune effector cell to mount an immune effector response. Examples of inhibitory molecules include PD-1, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGFR beta. Inhibition of a molecule that modulates or regulates, e.g., inhibits, T cell function, e.g., by inhibition at the DNA, RNA or protein level, can optimize an immune response. In embodiments, an agent (e.g., an inhibitory nucleic acid, such as a dsRNA, an siRNA or shRNA, a clustered regularly interspaced short palindromic repeats (CRISPR), a transcription-activator like effector nuclease (TALEN), or a zinc finger endonuclease (ZFN)) can be used to inhibit expression of an inhibitory molecule that inhibits the activity of the immune effector cell. In an embodiment, the inhibitor is an shRNA.

In one embodiment, the agent that modulates or regulates T-cell function can be an antibody or antibody fragment that binds to an inhibitory molecule. For example, the agent can be an antibody or antibody fragment that binds to PD-1, PD-L1, PD-L2 or CTLA4 (e.g., ipilimumab (also referred to as MDX-010 and MDX-101, and marketed as YERVOY™; Bristol-Myers Squibb; Tremelimumab (IgG2 monoclonal antibody available from Pfizer, formerly known as ticilimumab, CP-675,206). In an embodiment, the agent is an antibody or antibody fragment that binds to TIM3. In an embodiment, the agent is an antibody or antibody fragment that binds to LAG3.

Immune suppression can be reversed by inhibiting the local interaction of PD-1 with PD-L1. Antibodies, antibody fragments, and other inhibitors of PD-1, PD-L1 and PD-L2 are available in the art and may be used combination with a cars of the present disclosure described herein. For example, nivolumab (also referred to as BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449 and WO 06/121168. Pembrolizumab (formerly known as lambrolizumab, and also referred to as MK03475; Merck) is a humanized IgG4 monoclonal antibody that binds to PD-1. Pembrolizumab and other humanized anti-PD-1 antibodies are disclosed in U.S. Pat. No. 8,354,509 and WO 09/114335. MEDI4736 (Medimmune) is a human monoclonal antibody that binds to PDL1, and inhibits interaction of the ligand with PD1. MDPL3280A (Genentech/Roche) is a human Fc optimized IgG1 monoclonal antibody that binds to PD-L. MDPL3280A and other human monoclonal antibodies to PD-L1 are disclosed in U.S. Pat. No. 7,943,743 and US 2012/0039906. Other anti-PD-L1 binding agents include YW243.55.570 (heavy and light chain variable regions are shown in SEQ ID NOs 20 and 21 in WO 10/077634) and MDX-1 105 (also referred to as BMS-936559, and, e.g., anti-PD-L1 binding agents disclosed in WO 07/5874). AMP-224 (B7-DCIg; Amplimmune; e.g., disclosed in WO 10/27827 and WO 11/66342), is a PD-L2 Fc fusion soluble receptor that blocks the interaction between PD-1 and B7-H1. Other anti-PD-1 antibodies include AMP 514 (Amplimmune), among others, e.g., anti-PD-1 antibodies disclosed in U.S. Pat. No. 8,609,089, US 2010/028330, and US 2012/0114649.

TIM3 (T cell immunoglobulin-3) also negatively regulates T cell function, particularly in IFNγ-secreting CD4⁺T helper 1 and CD8⁺T cytotoxic 1 cells, and plays a critical role in T cell exhaustion. Antibodies, antibody fragments, and other inhibitors of TIM3 and its ligands are available in the art. Antibodies and peptides that inhibit TIM3 are disclosed in WO 13/6490 and US 2010/0247521. Other anti-TIM3 antibodies include humanized versions of RMT3-23 (disclosed in Ngiow et al. (2011) Cancer Res 71:3540-51), and clone 8B.2C12 (disclosed in Monney et al. (2002) Nature 415:536-41). Bi-specific antibodies that inhibit TIM3 and PD-1 are disclosed in US 2013/0156774.

LAG3 (lymphocyte activation gene-3 or CD223) is a cell surface molecule expressed on activated T cells and B cells that has been shown to play a role in CD8+ T cell exhaustion. Antibodies, antibody fragments, and other inhibitors of LAG3 and its ligands are available in the art. For example, BM S-986016 (Bristol-Myers Squib) is a monoclonal antibody that targets LAG3. IMP701 (Immutep) is an antagonist LAG3 antibody and IMP731 (Immutep and GlaxoSmithKline) is a depleting LAG3 antibody. Other LAG3 inhibitors include IMP321 (Immutep), which is a recombinant fusion protein of a soluble portion of LAG3. Other antibodies are disclosed in WO 10/19570.

Immune Modulating Molecules:

In certain embodiments, one or more immune modulating compounds can be administered as part of the treatment plan. Immune-modulating molecules comprise, but are not limited to cytokines, lymphokines, NK cell stimulating factors, and T cell co-stimulatory ligands. An immune-modulating molecule positively and/or negatively influences the humoral and/or cellular immune system, particularly cellular and/or non-cellular components, functions, and/or interactions with other physiological systems. Immune-modulating molecules may be selected from the group comprising cytokines, chemokines, macrophage migration inhibitory factor (MIF; as described, inter alia, in Bernhagen (1998) Mol Med 76(3-4):151-61), T-cell receptors or soluble MHC molecules. Such immune-modulating effector molecules are known in the art and are described, inter alia, in Paul, “Fundamental immunology”, Raven Press, New York (1989). Known cytokines and chemokines are described in Meager, “The Molecular Biology of Cytokines” (1998), John Wiley & Sons, Ltd.

Immune cell activity that may be measured include, but is not limited to, (1) cell proliferation by measuring the DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or the ability to respond to a chemotactin with chemotaxis; (8) immunosuppression, by inhibition of the activity of some other immune cell type; and, (9) apoptosis.

“Cytokine” herein refers to any factor produced by cells that affect cellular immunity. Examples of cytokines include but are not limited to IL-1 through IL-35, interferon alpha (IFN-α), IFN-β, IFNγ, tumor growth factor beta (TGF-β), TNF-α, and tumor necrosis factor beta (TNFβ).

Chemokines, similar to cytokines, are defined as any chemical factor or molecule which, when exposed to other cells, affects cellular immunity. Suitable chemokines may include but are not limited to the CXC, CC, C, and CX₃C chemokine families and to CCL-1 through CCL-28, CXC-1 through CXC-17, XCL-1, XCL-2, CX3CL1, MIP-1b, IL-8, MCP-1, and Rantes.

Growth factors include any molecules which when exposed to a particular cell induce proliferation and/or differentiation of the affected cell. Growth factors include proteins and chemical molecules including: stem cell factors, GM-CSF, G-CSF, human growth factor and stem cell growth factor. Additional growth factors may also be suitable for uses described herein.

Antiviral Therapy:

Compositions which include therapeutically effective amounts of at least one antiretroviral agent are also described herein. These composition can be administered sequentially or in conjunction with the ALT-803, IL-15, an IL-15 superagonist, interleukin-15 analogs, IL-15 mutant, or combinations thereof. In certain embodiments, the antiretroviral agent is a viral entry inhibitor, reverse transcriptase inhibitor, protease inhibitor, and/or immune-based therapeutic agent. For example, when used to treat or prevent HIV infection, the antiretroviral agent or its prodrug or pharmaceutically acceptable salt can be administered in combination or alternation with another anti-HIV agent and/or IL-15:IL-15Rα complex embodied herein. In general, in combination therapy, effective dosages of two or more agents are administered together, whereas during alternation therapy, an effective dosage of each agent is administered serially. Dosage will depend on absorption, inactivation and excretion rates of the drug, as well as other factors known to those of skill in the art. Dosage values also vary with the severity of the condition to be alleviated. For any particular subject, specific dosage regimens and schedules should be adjusted over time according to the individual need and the professional judgment of the one supervising administration of the compositions.

Combination therapy may be administered as a single pharmaceutical composition which comprises an antiretroviral agent as described herein, at least one ALT-803, IL-15, IL-15 superagonist, IL-15 mutant, or combinations thereof, and a pharmaceutically acceptable excipient, diluent, or carrier. Additionally or alternatively, combination therapy may be administered as two separate pharmaceutical compositions comprising (i) a first composition comprising an anti-retroviral agent as embodied herein and a pharmaceutically acceptable excipient, diluent, or carrier, and (ii) a second composition comprising at least one ALT-803, IL-15, IL-15 superagonist, IL-15 mutant, or combinations thereof as embodied herein. The pharmaceutical compositions can be administered simultaneously or sequentially and in any order.

In treating or preventing viral disease, the antiviral(s), e.g. antiretrovirals can be administered together with the IL-15:IL-15Rα complex, IL-15, an IL-15 superagonist, an IL-15 mutant, or combinations thereof as part of a unitary pharmaceutical composition. Alternatively, each can be administered apart from the other antiviral agents. In this embodiment, the antiretroviral(s) and the IL-15:IL-15Rα complex, IL-15, an IL-15 superagonist, IL-15 mutants or combinations thereof are administered substantially simultaneously, i.e. the compounds are administered at the same time or one after the other, so long as the compounds reach therapeutic levels for a period of time in the blood. In other embodiments, the antiretroviral agents are administered in one or more doses over a period of time followed by administration of the IL-15:IL-15Rα complex, IL-15, an IL-15 superagonist, IL-15 mutant, or combinations thereof.

The antiretroviral agents may be a nucleoside reverse transcriptase inhibitor, a nucleotide reverse transcriptase inhibitor, a non-nucleoside reverse transcriptase inhibitor, a protease inhibitor, an integrase inhibitor, a fusion inhibitor, a maturation inhibitor, or a combination thereof.

In certain embodiments, the at least one antiretroviral agent comprises: myristolyated dolutegravir, lamivudine, abacavir, rilpivirine or combinations thereof.

In certain embodiments, a composition comprises a therapeutically effective amount of IL-15:IL-15Rα complex and a non-nucleoside reverse transcriptase inhibitor (NNRTI) and/or a nucleoside reverse transcriptase inhibitor (NRTI), and/or myristolyated dolutegravir, lamivudine, abacavir, rilpivirine analogs, variants or combinations thereof. In certain embodiments, an NNRTI comprises: etravirine, efavirenz, nevirapine, rilpivirine, delavirdine, or nevirapine. In embodiments, an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate, or combinations thereof.

Examples of nucleoside reverse transcriptase inhibitors include zidovudine, didanosine, stavudine, zalcitabine, abacivir, emtricitabine, and lamivudine. Examples of non-nucleoside reverse transcriptase inhibitors include efavirenz, nevirapine, and delaviradine. Examples of protease inhibitors include indinavir, ritonavir, saquinavir, lopinavir, and nelfinavir. Examples of a reverse transcriptase inhibitor, an integrase inhibitor, a fusion inhibitor, and a maturation inhibitor are tenofovir, raltegravir, mariviroc, and bevirimat, respectively. In some aspects, the antiretroviral agents present in a nanoparticle include, ritonavir, lopinavir, and efavirenz, or efavirenz, abacavir, and lamivudine, or emtricitabine, tenofovir, and raltegravir.

In certain embodiments, the composition further comprises at least one or more protease inhibitors. In certain embodiments, a protease inhibitor comprises: amprenavir, tipranavir, indinavir, saquinavir mesylate, lopinavir and ritonavir (LPV/RTV), Fosamprenavir Calcium (FOS-APV), ritonavir, darunavir, atazanavir sulfate, nelfinavir mesylate, or combinations thereof.

In certain embodiments, the compositions comprise an anti-retroviral agent, used in HAART, chemotherapeutic agents, activators of HIV transcription, e.g. PMA, TSA, and the like. Antiretroviral agents may include reverse transcriptase inhibitors (e.g., nucleoside/nucleotide reverse transcriptase inhibitors, zidovudine, emtricitibine, lamivudine and tenoifvir; and non-nucleoside reverse transcriptase inhibitors such as efavarenz, nevirapine, rilpivirine); protease inhibitors, e.g., tipiravir, darunavir, indinavir; entry inhibitors, e.g., maraviroc; fusion inhibitors, e.g., enfuviritide; or integrase inhibitors e.g., raltegrivir, dolutegravir. Antiretroviral agents may also include multi-class combination agents for example, combinations of emtricitabine, efavarenz, and tenofivir; combinations of emtricitabine; rilpivirine, and tenofivir; or combinations of elvitegravir, cobicistat, emtricitabine and tenofivir.

In addition, one or more agents which alleviate any other symptoms that may be associated with the virus infection, e.g. fever, chills, headaches, secondary infections, can be administered in concert with, or as part of the pharmaceutical composition or at separate times. These agents comprise, without limitation, an anti-pyretic agent, anti-inflammatory agent, chemotherapeutic agent, or combinations thereof.

Some antiviral agents which can be used for combination therapy include agents that interfere with the ability of a virus to infiltrate a target cell. The virus must go through a sequence of steps to do this, beginning with binding to a specific “receptor” molecule on the surface of the host cell and ending with the virus “uncoating” inside the cell and releasing its contents. Viruses that have a lipid envelope must also fuse their envelope with the target cell, or with a vesicle that transports them into the cell, before they can uncoat.

There are two types of active agents which inhibit this stage of viral replication. One type mimics the virus-associated protein (VAP) and bind to the cellular receptors, including VAP anti-idiotypic antibodies, natural ligands of the receptor and anti-receptor antibodies, receptor anti-idiotypic antibodies, extraneous receptor and synthetic receptor mimics. The other type inhibits viral entry, for example, when the virus attaches to and enters the host cell. For example, a number of “entry-inhibiting” or “entry-blocking” drugs are being developed to fight HIV, which targets the immune system white blood cells known as “helper T cells”, and identifies these target cells through T-cell surface receptors designated “CRX4” and “CCR5”. Thus, CRX4 and CCR5 receptor inhibitors such as amantadine and rimantadine, can be used to inhibit viral infection, such as HIV.

Further antiviral agents that can be used in combination with the IL-15 compositions embodied herein, include agents that interfere with viral processes that synthesize virus components after a virus invades a cell. Representative agents include nucleotide and nucleoside analogs that look like the building blocks of RNA or DNA, but deactivate the enzymes that synthesize the RNA or DNA once the analog is incorporated. Acyclovir is a nucleoside analog, and is effective against herpes virus infections. Zidovudine (AZT), 3TC, FTC, and other nucleoside reverse transcriptase inhibitors (NRTI), as well as non-nucleoside reverse transcriptase inhibitors (NNRTI), can also be used. Integrase inhibitors can also be used.

Once a virus genome becomes operational in a host cell, it then generates messenger RNA (mRNA) molecules that direct the synthesis of viral proteins. Production of mRNA is initiated by proteins known as transcription factors, and certain active agents block attachment of transcription factors to viral DNA. Other active agents include antisense oligonucleotides and ribozymes (enzymes which cut apart viral RNA or DNA at selected sites). HIV include protease enzymes, which cut viral protein chains apart so they can be assembled into their final configuration. Protease inhibitors are another type of antiviral agent that can be used in combination with the inhibitory compounds described herein. The final stage in the life cycle of a virus is the release of completed viruses from the host cell.

Still other active agents function by stimulating the patient's immune system. Interferons, including pegylated interferons, are representative compounds of this class.

In certain embodiments, the anti-viral or antiretroviral agent comprises therapeutically effective amounts of: antibodies, aptamers, adjuvants, anti-sense oligonucleotides, chemokines, cytokines, immune stimulating agents, immune modulating molecules, B-cell modulators, T-cell modulators, NK cell modulators, antigen presenting cell modulators, enzymes, siRNA's, interferon, ribavirin, protease inhibitors, anti-sense oligonucleotides, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, vaccines, or combinations thereof.

Also of interest are enzymes present in the lytic package that cytotoxic T lymphocytes or LAK cells deliver to their targets. Perforin, a pore-forming protein, and Fas ligand are major cytolytic molecules in these cells. CTLs also express a family of at least 11 serine proteases termed granzymes, which have four primary substrate specificities. Low concentrations of streptolysin O and pneumolysin facilitate granzyme B-dependent apoptosis.

Other suitable effectors encode polypeptides having activity that is not itself toxic to a cell, but renders the cell sensitive to an otherwise nontoxic compound—either by metabolically altering the cell, or by changing a non-toxic prodrug into a lethal drug. Examples include thymidine kinase (tk), such as may be derived from a herpes simplex virus, and catalytically equivalent variants. The HSV tk converts the anti-herpetic agent ganciclovir (GCV) to a toxic product that interferes with DNA replication in proliferating cells.

Any of the above-mentioned compounds can be used in combination therapy with the IL-15:IL-15Rα complex, IL-15, an IL-15 superagonist, interleukin-15 analogs, IL-15 mutants or combinations thereof. Concurrent administration of two or more therapeutic agents does not require that the agents be administered at the same time or by the same route, as long as there is overlap in time during which the agents exert their therapeutic effect. Administration may be simultaneous or sequential, e.g., on different days or weeks. Therapeutic agents may be administered metronomically, i.e., continuous low-doses of a therapeutic agent.

Compositions described herein are suitable for use in a variety of drug delivery systems described above. Additionally, to enhance in vivo serum half-life, the compositions may be encapsulated, introduced into the lumen of liposomes, or prepared as a colloid. A variety of methods are available for preparing liposomes, as described in, e.g., U.S. Pat. Nos. 4,235,871, 4,501,728, & 4,837,028 each of which is incorporated herein by reference. Furthermore, one may administer the drug in a targeted drug delivery system, for example, in a liposome coated with a tissue specific antibody.

Appropriate compound doses are effective to prevent occurrence of the symptoms of the disorder or to treat some symptoms of the disorder from which the patient suffers. “Effective amount,” “therapeutic amount,” and “effective dose” each convey an amount sufficient to elicit the desired pharmacological or therapeutic effects, resulting in effective treatment of the disorder.

When treating viral infections, an effective amount of the inhibitory compound suppresses viral growth and proliferation. Viral infections can be prevented by administering the compounds described herein in a prophylactic manner. Preferably, the effective amount obtains the desired result without causing appreciable side effects.

Dosage, toxicity, and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects can be expressed as the ratio LD₅₀/ED₅₀.

Data obtained from cell culture assays and animal studies can be used in formulating a range of dosage for use in humans. The dosage of such compositions lies preferably within a range of circulating concentrations that include the ED₅₀ with little or no toxicity. Dosage may vary within this range depending upon the dosage form employed and the route of administration utilized. For any composition used in the methods described herein, the therapeutically effective dose can be estimated initially from cell culture assays. Doses may be formulated in animal models to achieve a circulating plasma concentration range that includes the IC₅₀ (i.e., the concentration of the test compound which achieves a half-maximal inhibition of symptoms) as determined in cell culture. Such information can be used to determine useful doses in humans. Plasma concentrations may be measured, for example, by high performance liquid chromatography.

As described, a therapeutically effective amount of a composition (i.e., an effective dosage) means an amount sufficient to produce a clinically desirable result. The compositions can be administered from one or more times per day to one or more times per week; including once every other day. Certain factors influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

Effective dose can vary, depending upon factors such as the condition of the patient, the severity of the viral infection, and the manner in which the pharmaceutical composition is administered. Effective dose differs from patient to patient, but in general includes amounts starting where desired therapeutic effects occur but below the amount where significant side effects are observed. For human patients, the effective dose of typical compounds generally requires administering the compound in an amount of at least about 1, often at least about 10, and frequently at least about 25 μg/24 hr/patient. The effective dose generally does not exceed about 500, often does not exceed about 400, and frequently does not exceed about 300 μg/24 hr/patient. In addition, administration of the effective dose is such that the concentration of the compound within the plasma of the patient normally does not exceed 500 ng/mL and frequently does not exceed 100 ng/mL. Effective dosing varies according to route of administration, the nature of the formulation, the nature of the patient's illness, patient size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2-, 3-, 4-, 6-, 8-, 10-, 20-, 50-, 100-, or 150-fold). Encapsulation of the compounds in a suitable delivery vehicle (e.g., polymeric microparticles or implantable devices) may increase the efficiency of delivery.

In some embodiments, the compositions may be formulated as a topical gel, for example, to treat a melanoma after excision, or an autoimmune condition expressed as a skin condition e.g. pemphigus. In some embodiments, the compositions can be formulated as a nanoparticle encapsulating a nucleic acid.

In methods of treatment of HIV-1 related cytopenia, a subject can be identified using standard clinical tests, for example, immunoassays to detect the presence of HIV antibodies or the HIV polypeptide p24 in the subject's serum, or through HIV nucleic acid amplification assays. In some methods, one can first determine whether a patient has a latent HIV infection, and then make a determination as to whether or not to treat the patient with a composition described herein.

The methods disclosed herein can be applied to a wide range of species, e.g., humans, non-human primates (e.g., monkeys), horses or other livestock, dogs, cats, ferrets or other mammals kept as pets, rats, mice, or other laboratory animals.

The methods disclosed herein can be expressed in terms of the preparation of a medicament. Accordingly, the invention encompasses the use of the agents and compositions described herein in the preparation of a medicament. The compounds described herein are useful in therapeutic compositions and regimens or for the manufacture of a medicament for use in treatment of diseases or conditions as described herein.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

Treatment duration can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. Treatment frequency can be variable. For example, the present compounds can be administered once (or twice, three times, etc.) daily, weekly, monthly, or yearly.

Formulation of Pharmaceutical Compositions: The compositions disclosed herein may be administered by any suitable means to achieve a concentration that, combined with other components, ameliorates, reduces, or stabilizes pancytopenia, in particular, lymphopenia. The composition may be provided in dosage forms suitable for parenteral (e.g., subcutaneous, intravenous, intramuscular, intravesicular, intratumoral or intraperitoneal) administration route. For example, the pharmaceutical compositions are formulated according to conventional pharmaceutical practice (see, e.g., Remington: The Science and Practice of Pharmacy (20th ed.).

Human dosage amounts are initially determined by extrapolating from the amount of compound used in mice or non-human primates, as a skilled artisan recognizes it is routine in the art to modify the dosage for humans compared to animal models. For example, the dosage may vary from between about 1 μg compound/kg body weight to about 5000 mg compound/kg body weight; or from about 5 mg/kg body weight to about 4,000 mg/kg body weight or from about 10 mg/kg body weight to about 3,000 mg/kg body weight; or from about 50 mg/kg body weight to about 2000 mg/kg body weight; or from about 100 mg/kg body weight to about 1000 mg/kg body weight; or from about 150 mg/kg body weight to about 500 mg/kg body weight. For example, the dose is about 1, 5, 10, 25, 50, 75, 100, 150, 200, 250, 300, 350, 400, 450, 500, 550, 600, 650, 700, 750, 800, 850, 900, 950, 1,000, 1,050, 1,100, 1,150, 1,200, 1,250, 1,300, 1,350, 1,400, 1,450, 1,500, 1,600, 1,700, 1,800, 1,900, 2,000, 2,500, 3,000, 3,500, 4,000, 4,500, or 5,000 mg/kg body weight. Alternatively, doses are in the range of about 5 mg compound/kg body weight to about 20 mg compound/kg body weight. In another example, the doses are about 8, 10, 12, 14, 16 or 18 mg/kg body weight. Where ALT-803 is administered as part of the therapy, the ALT-803 is administered at 0.5 mg/kg to ˜10 mg/kg (e.g., 0.5, 1, 3, 5, 10 mg/kg). This dosage amount may be increased or decreased depending on results of the needs of a particular patient.

Pharmaceutical compositions are formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes. The pharmaceutical compositions embodied herein may be administered parenterally by injection, infusion, or implantation (subcutaneous, intravenous, intramuscular, intratumoral, intravesicular, intraperitoneal) in dosage forms, formulations, or via suitable delivery devices or implants containing conventional, non-toxic pharmaceutically acceptable carriers and adjuvants. Suitable formulations can be found in Remington, supra.

Compositions comprising ALT-803 for parenteral use may be provided in unit dosage forms (e.g., in single-dose ampoules). Alternatively, ALT-803 may be provided in vials containing several doses, and in which a suitable preservative may be added (see below). The composition may be in solution, suspension, emulsion, infusion device, or a delivery device for implantation. ALT-803 may be provided as dry powder to be reconstituted with water or another suitable vehicle before use. Apart from the active agent that reduces or ameliorates a neoplasia or infectious disease, the composition includes suitable parenterally acceptable carriers and/or excipients. The active therapeutic agent(s) may be incorporated into microspheres, microcapsules, nanoparticles, liposomes for controlled release. Furthermore, the composition may include suspending, solubilizing, stabilizing, pH-adjusting agents, tonicity adjusting agents, and/or dispersing, agents.

Pharmaceutical compositions may be suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol.

Methods of treating pancytopenia are described herein, comprising administering a therapeutically effective amount of a pharmaceutical composition. Thus, one embodiment is a method of treating a subject suffering from or susceptible to lymphopenia. The method includes the step of administering to the mammal a therapeutic amount of the compositions embodied herein, in a dose sufficient to treat the disease or disorder or symptom thereof, under conditions such that the disease or disorder is treated.

Methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method). A subject (e.g., animal, human) in need thereof, may be a mammal, particularly a human. Treatment will be suitably administered to subjects, particularly humans, suffering from, having, susceptible to, or at risk for a neoplasia, infectious disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like).

Methods described herein may also include the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with neoplasia in which the subject has been administered a therapeutic amount of a compound herein sufficient to treat the disease or symptoms thereof. Marker levels determined in the method can be compared to known levels of Marker in either healthy normal controls or in other afflicted patients to establish the subject's disease status. In some cases, a second level of Marker in the subject is determined at a time point later than the determination of the first level, and the two levels are compared to monitor the course of disease or the efficacy of the therapy. In certain aspects, a subject's pre-treatment level of Marker is determined prior to treatment; this pre-treatment Marker level can then be compared to the Marker level in the subject after the treatment commences, to determine treatment efficacy.

Kits: The compositions described herein can be packaged in suitable containers labeled, for example, for use as a therapy to treat a subject having pancytopenia. The containers can include a composition comprising an IL-15:IL-15Rα complex, IL-15, an IL-15 superagonist, IL-15 mutant, or combinations thereof, along with a second therapeutic agent and one or more of a suitable stabilizer, carrier molecule, flavoring, and/or the like, as appropriate for the intended use. Accordingly, packaged products (e.g., sterile containers containing one or more of the compositions described herein and packaged for storage, shipment, or sale at concentrated or ready-to-use concentrations) and kits, including instructions for use, are also disclosed herein. A product can include a container (e.g., a vial, jar, bottle, bag, or the like) containing one or more compositions disclosed herein. In addition, an article of manufacture further may include, for example, packaging materials, instructions for use, syringes, delivery devices, buffers or other control reagents for treating or monitoring the condition for which prophylaxis or treatment is required.

The kit may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions can be provided in a concentrated form with a diluent and instructions for dilution.

While various embodiments of the composition and methods disclosed herein have been described above, it should be understood that they have been presented by way of example only, and not limitation. Numerous changes to the disclosed embodiments can be made in accordance with the disclosure herein. Thus, the breadth and scope of the claimed invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, applicants do not admit any particular reference is “prior art” to their invention.

### EXAMPLES

### Example 1: A Pharmacokinetic Study of Subcutaneous ALT-803

Materials and Methods

Ethical Conduct of the Study:

The study was conducted in full compliance with the principles of the “Declaration of Helsinki” (as amended in Tokyo, Venice, Hong Kong, and South Africa), International Conference on Harmonisation (ICH) guidelines, and all of the applicable United States (US) Code of Federal Regulations (CFR), 21 CFR Part 50 & 312.

Subject Information and Consent:

Informed consent was obtained and documented in compliance with applicable regulatory requirement(s) and good clinical practice (GCP), as well as ethical principles having their origin in the Declaration of Helsinki. Prior to beginning the study, the investigator had the IRB/IECs written approval of the written informed consent form and any other information provided to subjects.

Non-Clinical Pharmacokinetics.

When administered to CD-1 mice as a single IV injection of 1 mg/kg, ALT-803 had an estimated half-life of about 18 hr (IgG1 domain assay) to 25 hr (full complex assay). These results indicate that the protein complex was not cleaved and IL-15N72D did not significantly dissociate from the IL-15RαSu/IgG1-Fc molecule in vivo. The clearance (Cl) of ALT-803 ranged from 0.059 to 0.051 mL/h and the volume of distribution at steady state (Vss) ranged from 2.1 to 1.3 mL depending on the assay format. In comparison, IL-15 had an absorption half-life of 0.24 hr and a terminal half-life of 0.64 hr. The Cl of IL-15 was 49 mL/h, and the Vss was 18.4 mL. These results indicate that ALT-803 displays a >24-fold longer terminal half-life and is cleared >800-fold slower than IL-15 in mice.

In cynomolgus monkeys, a dose dependent increase in systemic exposure to ALT-803 followed the IV administration of 0.03 and 0.10 mg/kg ALT-803. ALT-803 terminal elimination half-life was approximately 7.6 hr in cynomolgus monkeys and did not appear to significantly differ between dose levels. The maximum serum concentration (Cₘₐₓ) for the 0.1 mg/kg ALT-803 group is consistent with full recovery of the administered dose, whereas Cₘₐₓ and AUC_{0-inf} parameters suggest that there is ˜30% less recovery at the 0.03 mg/kg dose. However, even at the low dose level, the Cₘₐₓ of ALT-803 in the serum was over 400-fold the EC₅₀ value (˜16 μM) determined for ALT-803 stimulated proliferation of IL-15RβγC-bearing cells. The steady state volume of distribution ranged between 35 and 55 mL/kg as expected based on the plasma volume in the vasculature.

ALT-803.

Multiple clinical trials are currently being conducted using ALT-803 in subjects with various cancer indications including both hematologic malignancies and solid tumors. Over 200 subjects have received multiple doses of ALT-803 as a single agent or in combination with other immunotherapeutic agents such as BCG (Bacillus Calmette-Guerin), rituximab, and nivolumab. ALT-803 has been administered by intravesical instillation, IV infusion and subcutaneous injection, and has been administered in doses ranging from 0.1 μg/kg to 20 μg/kg. The most common adverse event attributed to ALT-803 administered subcutaneously is an injection site reaction. Injection site reactions are localized rashes surrounding the ALT-803 injection site, and are attributed to a dermal infiltrate of immune cells comprised of mostly CD4⁺ and CD8⁺ T cells, as well as NK cells and macrophages. Injection site reactions typically occur 3 days post ALT-803 subcutaneous injection and resolve in less than one week.

Subcutaneous ALT-803 has been administered in phase ½ studies starting at a dose of 0.3 μg/kg, escalated up to a dose of 20 μg/kg. ALT-803 is provided in a 2 mL vial containing 1.2 mL of ALT-803 at 1.0 mg/mL and in a 2 mL vial containing 0.6 mL at 2.0 mg/mL.

Overall Study Design.

This was a single center, open-label, pharmacokinetic study of ALT-803 administered as a subcutaneous injection to healthy subjects. 16 subjects meeting the entry criteria were randomized in a 1:1 ratio (Groups A or B) to one of two ALT-803 drug concentrations. Four out of 6 subjects who discontinued prior to Study Period 2, met criteria for replacement and were replaced. Therefore, total enrolment for this study was 20 subjects. Subjects randomized to Group A (n=8) received ALT-803 at a concentration of 1.0 mg/mL and subjects randomized to Group B (n=8) received ALT-803 at a concentration of 2.0 mg/mL. Subjects received a single 10 μg/kg subcutaneous dose of ALT-803 on Day 1 of Study Period 1. After a rest period, subjects received a single 20 μg/kg subcutaneous dose of ALT-803 on Day 1 of Study Period 2. Blood samples to determine serum levels of ALT-803 were collected prior to dosing, and at 1, 4, 24, 48, 72, 96, 120, 144, 168 and 192 hours after dosing. Vital signs (heart rate, blood pressure, respiration, temperature) were monitored.

Safety was assessed for all subjects, including monitoring of vital signs and incidence and severity of adverse events (AEs). Blood samples were collected for hematology and chemistry, immune cell levels and activity, immune cell phenotype subsets, cytokine levels and immunogenicity testing, which include assays for anti-ALT-803 antibodies.

After Study Period 2 finished, subjects were followed for an additional 6 days and had an End of Study visit on Day 15. This included safety labs, immune cells, and cytokines. Concomitant medications and adverse events were collected throughout the study. The study schematic is shown in FIG. 1.

Selection of Study Population-Inclusion Criteria.

In order to be considered eligible, all of the following criteria must have been met:

1. Signed Written Informed Consent
a. Subjects must have signed and dated an IRB/IEC approved written informed consent form in accordance with regulatory and institutional guidelines. This must be obtained before the performance of any protocol related procedures that are not part of subject care.
b. Subjects must be willing and able to comply with the scheduled visits, study drug dosing schedule, procedures, laboratory tests, and other requirements of the study.

2. Study Population
a. Body mass index (BMI) must be within the range of 18 to 28 kg/m². Subjects must weigh between 50 and 100 kg (inclusive).
b. Subjects must be in good health as determined by past medical history, complete physical examination, vital signs and laboratory tests at screening.

3. Age and Reproductive Status
a. Men and women, 18-65 years of age.
b. Female participants of childbearing potential must adhere to using a medically accepted method of birth control up to 28 days prior to screening and agree to continue its use during the study or be surgically sterilized (e.g., hysterectomy or tubal ligation) WOCBP must agree to use effective contraception during the study and for at least 1 month following the last dose of the study drug.
c. WOCBP must have a negative serum pregnancy test <14 days prior to first dose of the study drug. Non-childbearing is defined as greater than one year postmenopausal or surgically sterilized.
d. Male subjects must be willing to use barrier contraception (i.e. condoms and spermicide) from the day of dosing until at least 1 month following the last dose of study drug.

Exclusion Criteria.

Subjects who met any one of the following criteria were ineligible for participation in the study:

1. Medical History and Concurrent Diseases
a. A past medical history of clinically significant 12 lead EKG abnormalities
b. Subjects with a history of interstitial lung disease and/or pneumonitis.
c. HIV-positive.
d. Significant illness within 2 weeks prior to dosing.
e. Positive hepatitis C serology or active hepatitis B infection.
f. Known autoimmune disease requiring active treatment. Subjects with a condition requiring systemic treatment with either corticosteroids (>10 mg daily prednisone equivalent) or other immunosuppressive medications within 4 weeks or 5 half-lives of registration are excluded.
g. Psychiatric illness/social situations that would limit compliance with study requirements.
h. Previous malignancies, unless basal or squamous cell carcinoma of the skin or cervical carcinoma in situ with a complete remission achieved at least 5 years prior to study entry and no additional therapy is required or anticipated to be required during the study period.
i. Loss of ≥475 mL blood volume or blood donation transfusion of any blood product within 3 months prior to screening.
j. Other illness or laboratory abnormality that in the opinion of the Investigator should exclude the subject from participating in this study.

2. Prohibited Treatments and/or Restricted Therapies
a. Use of any prescription drugs within 4 weeks (hormonal methods of contraception are allowed) or less than 5 half-lives prior to dosing, or over-the-counter (OTC) medication (vitamins, herbal supplements, dietary supplements) within 2 weeks or less than 5 half-lives prior to dosing.
b. Exposure to any investigational drug or placebo within 3 months of first dose of study drug.
c. Previous treatment or clinical trial participation with monoclonal antibody therapy.
d. History of drug or alcohol abuse within 12 months prior to dosing, or those who have a positive urine drug test or breath alcohol test at Screening or Baseline.
e. Transfusion of blood or any blood product within 3 months prior to screening.
f. History of using nicotine-containing products or smoking more than 5 cigarettes weekly for at least three months prior to the study through the final evaluation.

3. Allergies and Adverse Drug Reaction
a. History of severe hypersensitivity reactions to other monoclonal antibodies.
b. Known history of clinically significant drug allergy at Screening or Baseline

4. Sex and Reproductive Status
a. Women who are pregnant or nursing.

Removal of Subjects from Therapy or Assessment.

All subjects had the right to withdraw formal consent without prejudice at any time during the study. If a subject withdrew formal consent, the investigator was to make a reasonable effort to determine the cause for withdrawal of consent. For these subjects, as well as all other subjects who required permanent discontinuation of study drug, the investigator was to make a reasonable effort to complete all required study procedures. Of the 16 subjects that were enrolled, two subjects were withdrawn from study due to AEs (0317-067-013 and 0317-067-0011). Four subjects met replacement criteria and were replaced. Three of the four subjects that were replaced (0317-067-003, 0317-067-0008, and 0317-067-014) voluntarily withdrew consent; and, the fourth subject (0317-067-0015) was replaced due to a protocol deviation. Any subject who met the following criteria was to be replaced:
Subjects who are enrolled but did not receive ALT-803
Subjects who withdrew consent
Subjects who had a positive alcohol or drug test after enrollment
Subjects who missed more than one PK collection timepoint during Study Period 1 or Study Period 2.

Subjects who were replaced were to be followed for up to 14 days, following their last dose of ALT-803 for adverse events monitoring only.

Treatment(s) Administered.

ALT-803 was provided in a 2 mL vial containing 1.2 mL of ALT-803 at 1 mg/mL and a 2 mL vial containing 0.6 mL of ALT-803 at 2 mg/mL. Vials were packaged in cartons and are delivered to the research site.

There were 2 groups of subjects, Group A and Group B. At enrollment, but prior to first dose, each group was assigned to receive one of the two ALT-803 drug concentrations (Group A at 1.0 mg/mL and Group B at 2.0 mg/mL). Both groups received a single subcutaneous injection of ALT-803 at a dose of 10 μg/kg on Day 1 and crossed over to a dose of 20 μg/kg on Day 15.

Dose calculations were based on subject's assigned dose level and actual body weight collected prior to ALT-803 dose for each study period. The calculated amount of ALT-803 was drawn into a syringe for subcutaneous injection. If the total subcutaneous dose was greater than 1.5 mL, the dose would be divided into 2 or 3 subcutaneous injections as needed. Injection sites were rotated for Study Period 2, per institutional guidelines and each injection site (preferably the abdomen) separated by at least 1 inch.

Identity of Investigational Product(s).

ALT-803 is a soluble complex consisting of two protein subunits of a human IL-15 variant associated with high affinity to a dimeric human IL-15 alpha receptor sushi domain/human IgG1 Fc fusion protein. The human IL-15Rα sushi domain/human IgG1 Fc fusion protein comprises the sushi domain of the human IL-15 receptor α subunit (IL-15Rα) (aa 1-65 of the mature human IL-15Rα protein) linked with the human IgG1 CH2-CH3 region containing the Fc domain (232 amino acids). Aside from the N72D substitution, all of the protein sequences are human. ALT-803 must be maintained at a temperature between 2° C. and 8° C. and this was verified at the research site by the appointed Altor monitor. Stability studies have been conducted on IP diluted in 0.9% saline. The results of the studies showed that ALT-803 potency was maintained within the specifications when diluted in 0.9% saline to a concentration of 25 μg/mL and stored in a syringe for <24 hours at 4° C.

Method of Assigning Subjects to Treatment Groups.

This was a single center and open-label study. Subjects meeting the entry criteria were randomized in a 1:1 ratio (Groups A or B) to one of two ALT-803 drug concentrations. A total of 16 evaluable subjects were enrolled (8 in Group A and 8 in Group B).

Selection of Doses in the Study.

Subjects randomized to Group A (n=10) received 1.0 mg/mL ALT-803 and subjects randomized to Group B (n=10) received 2.0 mg/mL ALT-803. Groups A and B received a single 10 μg/kg subcutaneous dose of ALT-803. After a Rest Period, groups A and B received a single 20 μg/kg subcutaneous dose of ALT-803. Results from recently completed and ongoing studies in oncology support therapeutics doses of ALT-803 of 10 μg/kg for hematologic malignancies and up to 20 μg/kg in solid tumor malignancies.

Selection and Timing of Dose for Each Subject.

Subjects received an initial single 10 μg/kg subcutaneous dose of ALT-803. After a Rest Period of at least 6 days, subjects received a single 20 μg/kg subcutaneous dose of ALT-803.

Prior and Concomitant Therapy.

At screening, use of any prescription drugs were prohibited within 4 weeks (hormonal methods of contraception were allowed) or less than 5 half-lives prior to dosing. over-the counter (OTC) medications were prohibited within 2 weeks or less than 5 half-lives prior to dosing were not permitted. Concomitant medications [prescription drugs and OTC medications (vitamins, herbal supplements, analgesics)] were not permitted while on study, with the exception of topical 0.05% clobetasol propionate (i.e. 0.05% Cormax) or 0.1% triamcinolone (i.e., Kenalog) cream; and, Diphenhydramine could be administered pre-(25-50 mg orally) and post-dosing (25-50 mg TID orally as needed) of ALT-803 at the discretion of the Investigator. Concomitant medications were assessed on a continual basis starting on Day 1, until the last study visit.

Treatment Compliance.

All study drug was administered by the study investigator or designated member of staff at the clinical research site. To ensure drug accountability the investigator or designated deputy maintained accurate records of the dates and amounts of drug received, to whom it was dispensed and accounts of any supplies which were accidentally or deliberately destroyed; these details were recorded on a drug accountability form. All unused clinical supplies and the drug accountability forms were returned to Altor BioScience at the end of the study. The protocol required complete adherence to safeguard subject wellbeing and ensure data integrity. Noncompliance could result in a deviation or subject replacement. The Investigator was responsible for ensuring that the study was conducted in accordance with the procedures described in the protocol and was prohibited from implementing any changes to the protocol unless it was required to eliminate an immediate hazard to the subject. If a deviation occurred that affected the safety of a subject, Altor BioScience was to be notified immediately.

### Pharmacokinetic and Safety Variables

Screening and Baseline (Day −14 Through Time 0).

Time 0 was defined as the start of the first study treatment administration. Baseline was defined as Study Day 1 prior to the first dose of study treatment (Time 0). The Screening Period was the 14-day period prior to Baseline.

Study Period 1, Day 1-Day 9.

Study period 1 was defined as the 9 days following Time 0, Day 1. In Study Period 1, subjects were administered a single subcutaneous injection of ALT-803. Blood sampling for PK, immune cells, cytokines and safety labs commenced on Day 1, and continued daily throughout the nine-day study period.

Rest Period (>6 Days).

No study visits occurred during the rest period. Monitoring for adverse events and use of concomitant medications continued throughout this period.

Study Period 2, Day-Day 9.

Study period 2 was defined as the 9 days following the Rest period. In Study Period 2, subjects were administered a single subcutaneous injection of ALT-803. For Study Period 2, blood sampling for PK, immune cells, cytokines and safety labs commenced on Day 1, and continued daily throughout the nine-day study period.

Follow-up: Study Period 2, Day 10-Day 15.

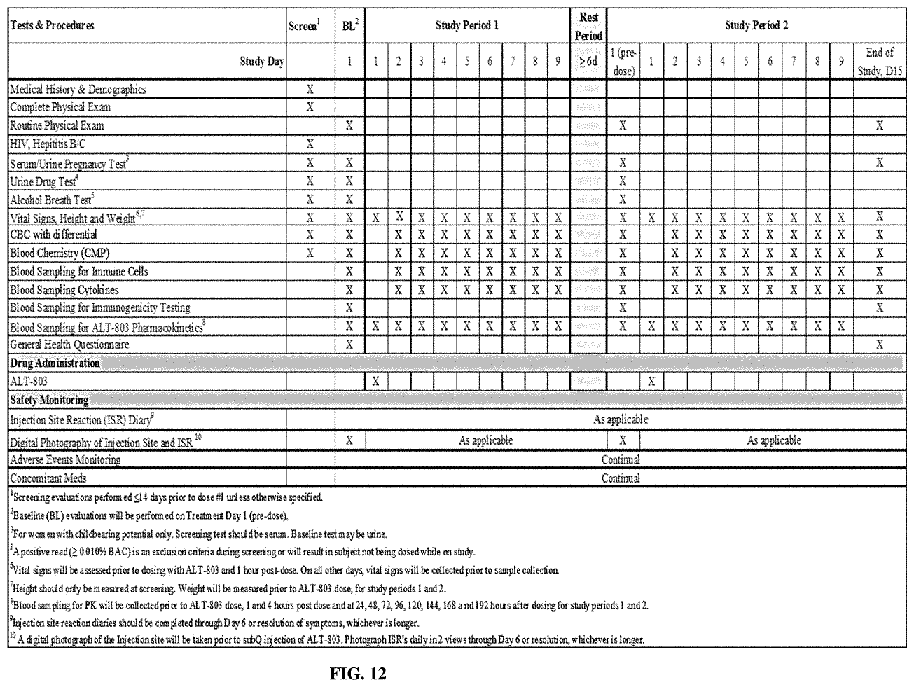

The follow-up period was defined as the 6-day period following the completion of Study Period 2. Monitoring for adverse events and use of concomitant medications continued throughout this period. Subjects were asked to return to the clinical research site on Day 15 for an End of Study visit. The schedule of assessments is presented as FIG. 12.

Pharmacokinetic Parameters.

After reviewing the available PK data, the following PK parameters were calculated from the serum concentration-time data using standard non-compartmental pharmacokinetic methods and the computer program Phoenix 64 Build 8.0.0.3176 (Certara LP).
Tₘₐₓ—Time to maximum concentration since last dose during Study Periods 1 and 2
Cₘₐₓ—observed concentration during Study Periods 1 and 2
T_{1/2}—apparent terminal half-life during Study Periods 1 and 2
AUC₀₋₇₂—Partial Area under the curve time 0-72 hours during Study Periods 1 and 2
AUC_{0-inf}—Area under the plasma concentration curve from time 0 extrapolated to infinite time for Study Periods 1 and 2
Vz/F—apparent (extravascular) volume of distribution for Study Periods 1 and 2
CL/F—Apparent (extravascular) clearance for Study Periods 1 and 2

No statistical comparisons were performed for the PK parameters. All PK parameters were analyzed descriptively including mean and standard deviation.

Secondary Endpoints.

The secondary endpoint of the study was to assess safety as measured by the incidence and severity of adverse events.

Measures of Interest.

Blood samples were collected for immune cell levels and activity, immune cell phenotype subsets, cytokine levels and immunogenicity testing, which included assays for anti-ALT-803 antibodies. General health was assessed by the RAND General Health Questionnaire (SF-36).

Statistical and Analytical Methods

Analysis Populations.

The analysis populations were defined as follows:

Safety population: Safety population includes all enrolled subjects who received at least one dose of ALT-803.

PK population: PK population included all subjects who received both doses of ALT-803, had any reported ALT-803 concentrations, and no more than one missing PK sample during each study period. These subjects were used in the tabulated concentration summaries and listings as well as the individual and mean graphs. The PK Analysis population included all subjects that had sufficient measurable concentrations to support PK parameter analysis. These subjects were used in the tabulated PK summaries as well as the individual PK parameter listings.

All protocol deviations that occurred during the study were considered for severity/impact and were taken into consideration when subjects were assigned to the PK analysis population.

General Methodology.

Statistical analyses are descriptive in nature. Descriptive statistics consist of the number and percentage of subjects in each category for discrete variables, and the sample size, mean, median, S.D., minimum, and maximum for continuous variables. All mean and median values are formatted to one more decimal place than the measured value. Standard deviation values are formatted to two more decimal places than the measured value. Confidence intervals (CIs) are presented as 2-sided 95% Cis. The day of the first dose of any study drug was defined as Day 1. Baseline value was defined as the last value before the first dose of any study drug is administered.

Demographic and Baseline Characteristics.

Summary statistics were calculated for age, sex, ethnicity, race, height, and baseline body weight. Age was defined as the time from the subject's date of birth to date of informed consent in years.

AEs.

Several summary tables were presented for the incidence of treatment-emergent adverse events (TEAEs). TEAEs were defined as any AE that begins or worsens in grade after the start of study drug until 30 days after the last dose of study drug or End of Study, whichever is later.

Summary tables were tabulated alphabetically by SOC and PT based on the MedDRA version 19.1 coding dictionary. If a subject experienced multiple episodes of the same AE, the subject only would be counted once, for that particular AE. Any AE with a missing onset date was considered as treatment-emergent unless there was a non-missing end date information (i.e., month or year) that indicated it resolved prior to administration of study drug. If a subject experienced multiple episodes of the same event, the event with the maximum severity or strongest relationship to study medication was used for analysis. Missing severity or relationship were presented as “Severe” or “Suspected”, respectively. SAEs, deaths, and AEs leading to discontinuation of study drug were presented in data listings. Any non-TEAEs were presented in the data listings and indicated as non-TEAE.

Ital Signs and Physical Examinations.

Vital signs (heart rate, blood pressure, respiratory rate, body temperature) were collected at screening and at study visits. Vital signs were performed prior to, and one-hour post-ALT-803 subcutaneous injection. On PK sample collection days, vital signs were collected prior to blood draw. Height was measured at screening and body weight noted before each ALT-803 subcutaneous injection.

A complete physical exam [head, eyes, ears, nose, throat, skin, heart, lungs, abdomen, extremities, & neurological (e.g. level of consciousness, pupils, motor/sensory responses, & reflexes)] was performed at screening and a routine physical exam was performed on all study visits. Vital signs and body weight was summarized at baseline and as change from baseline at each post-baseline evaluation time point. Abnormal vital signs and physical examination findings were reported as AEs.

Injection Site Reaction.

The most common adverse event attributed to ALT-803 administered subcutaneously is an injection site reaction. Injection site reactions are localized rashes surrounding the ALT-803 injection site. Injection site reactions typically occur 3 days post ALT-803 subcutaneous injection and resolve in less than one week.

The ALT-803 Injection Site Reaction Diary was given to subjects on the day that the ALT-803 subcutaneous injection is administered (Day 1 of Study Periods 1 and 2). The diary was completed by the subject daily until the resolution of symptoms. If any symptoms had not resolved by the next dose of ALT-803 (Day 1 of Study Period 2), the subject needed to complete two diaries. Each diary addressed the individual injection sites and their corresponding symptoms until resolution. The subject returned the completed diaries to the study coordinator. Any of the symptoms that represent an adverse event was reported as an AE.

### Study Population

Disposition of Subjects.

A total of 20 subjects were enrolled and included in the safety population. Of the 20 subjects enrolled, 14 completed the study and six discontinued the study. Reasons for discontinuation included withdrawal by subject (n=3), adverse event (n=2) and protocol deviation (n=1). A summary of Subject Disposition is summarized below in Table 1 and displayed in FIG. 2 Data Sets Analyzed.

The Safety Population (n=20) includes all enrolled subjects who received at least one ALT-803 dose. The PK Population (n=14) includes all subjects who received both ALT-803 doses, had any reported ALT-803 concentrations, and no more than one missing PK sample during each study period.

TABLE 1
Subject Disposition: All Enrolled Subjects

| | ALT-803 | | ALT-803 | | All | |
|---|---|---|---|---|---|---|
| | 1.0 mg/mL | | 2.0 mg/mL | | Subjects | |
| | n = 10 | | n = 10 | | n = 20 | |
| Safety Population, n (%) | 10 | (100%) | 10 | (100%) | 20 | (100%) |
| Study Completed | 7 | (70%) | 7 | (70%) | 14 | (70%) |
| Study Discontinued | 3 | (30%) | 3 | (30%) | 6 | (30%) |
| Safety Population, n (%) | 10 | (100%) | 10 | (100%) | 20 | (100%) |
| Reasons for Discontinuation, | | | | | | |
| n (%): | | | | | | |
| Adverse Event | 1 | (33%) | 1 | (33%) | 2 | (33%) |
| Protocol Deviation | 1 | (33%) | 0 | | 1 | (17%) |
| Withdrawal by Subject | 1 | (33%) | 2 | (67%) | 3 | (50%) |

Demographics and Other Baseline Characteristics.

The mean age of subjects enrolled in the study is 41.5±11.35 years (range 23-57 years). The majority of subjects were white (95%), Hispanic or Latino (95%) and 11 (55%) were male and 9 (45%) were female. The mean weight of subjects was 71.6±9.5 kg (range 50.3-84.9 kg). A summary of demographics by group and overall for the Safety population is summarized in in Table 2.

TABLE 2
Demographics and Baseline Characteristics
(Population: Safety Population)

| | ALT-803 | ALT-803 | | All | |
|---|---|---|---|---|---|
| | 1.0 mg/mL | 2.0 mg/mL | | Subjects | |
| | n = 10 | n = 10 | | n = 20 | |
| Age (years) | | | | | |
| Mean | 43.7 | 39.3 | | 41.5 | |
| SD | 11.29 | 11.57 | | 11.35 | |
| Min, Max | 26, 57 | 23, 57 | | 23, 57 | |
| Gender | | | | | |
| Male | 5 (50%) | 6 | (60%) | 11 | (55%) |
| Female | 5 (50%) | 4 | (40%) | 9 | (45%) |
| Ethnicity | | | | | |
| Hispanic or Latino | 9 (90%) | 10 | (100%) | 19 | (95%) |
| Not Hispanic or Latino | 1 (10%) | 0 | | 1 | (5%) |
| Race | | | | | |
| Black or African American | 1 (10%) | 0 | | 1 | (5%) |
| White | 9 (90%) | 10 | (100%) | 19 | (95%) |

| Weight (kg) | | | |
|---|---|---|---|
| Mean | 69.92 | 73.24 | 71.58 |
| SD | 7.66 | 11.21 | 9.50 |
| Min, Max | 57.6, 83.6 | 50.3, 84.9 | 50.3, 84.9 |

Pharmacokinetic and Pharmacodynamic Results:

The PK Population (n=14) includes all subjects who received both doses of ALT-803, had any reported ALT-803 concentrations, and no more than one missing PK sample during each study period.

Plasma Sample Collection.

In Study Period 1, subjects were administered a single subcutaneous injection of 10 μg/kg ALT-803. Blood samples to determine serum levels of ALT-803 were collected prior to dosing, and at 1, 4, 24, 48, 72, 96, 120, 144, 168 and 192 hours after dosing (nine-day Study Period 1). A rest period of at least six days followed Study Period 1. In Study Period 2, subjects were administered a single subcutaneous injection of 20 μg/kg ALT-803 and blood samples to determine serum levels of ALT-803 were again collected prior to dosing, and at 1, 4, 24, 48, 72, 96, 120, 144, 168 and 192 hours after dosing (nine-day Study Period 2).

Bioanalysis of Plasma Samples.

Serum concentrations for ALT-803 were determined using ELISA assay. For quantitative determination of human IL-15 in serum, plasma and cell culture supernatant, the R&D Systems Quantikine ELISA Human IL-15 Immunoassay Kit was developed by R&D Systems. The assay uses a solid phase sandwich ELISA method employing anti IL-15 antibodies as both the capture and detection antibodies.

Pharmacokinetic and Pharmacodynamic Analysis.

The evaluable population (for pharmacokinetics) included all subjects who had received both doses of ALT-803 and had no more than one missing PK sample during each study period. Therefore, all 14 from the PK population were included in the PK analysis and this subset of analysis was termed as PK Population. However PK samples were also collected during Study Period 1 for an additional 6 subjects bringing the total to n=20 and this subset of analysis was termed as Safety Population.

Following PK parameters were calculated from the serum concentration-time data using standard non-compartmental pharmacokinetic methods and the computer program Phoenix 64 Build 8.0.0.3176 (Certara LP).
Tₘₐₓ—Time to maximum concentration since last dose during Study Periods 1 and 2
Cₘₐₓ—observed concentration during Study Periods 1 and 2
T_{1/2}—apparent terminal half-life during Study Periods 1 and 2
AUC₀₋₇₂—Partial Area under the curve time 0-72 hours during Study Periods 1 and 2
AUC_{0-inf}—Area under the plasma concentration curve from time 0 extrapolated to infinite time for Study Periods 1 and 2
Vz/F—apparent (extravascular) volume of distribution for Study Periods 1 and S 2
CL/F—Apparent (extravascular) clearance for Study Periods 1 and 2

No statistical comparisons were performed for the PK parameters. All PK parameters were analyzed descriptively including mean and standard deviation.

ALT-803 Plasma Concentrations:

The pharmacokinetics of ALT-803 upon subcutaneous administration were represented by a typical absorption profile followed by biphasic disposition with an initial rapid distribution phase and an elimination phase. Mean concentration data for all subjects was within 43-277% CV due to unusually high concentrations reported for subject 0317-001-0020 during Study Period 2. Dosing records for the subject did not indicate any dosing errors and a reassay was performed to confirm the concentrations.

Pharmacokinetic Results:

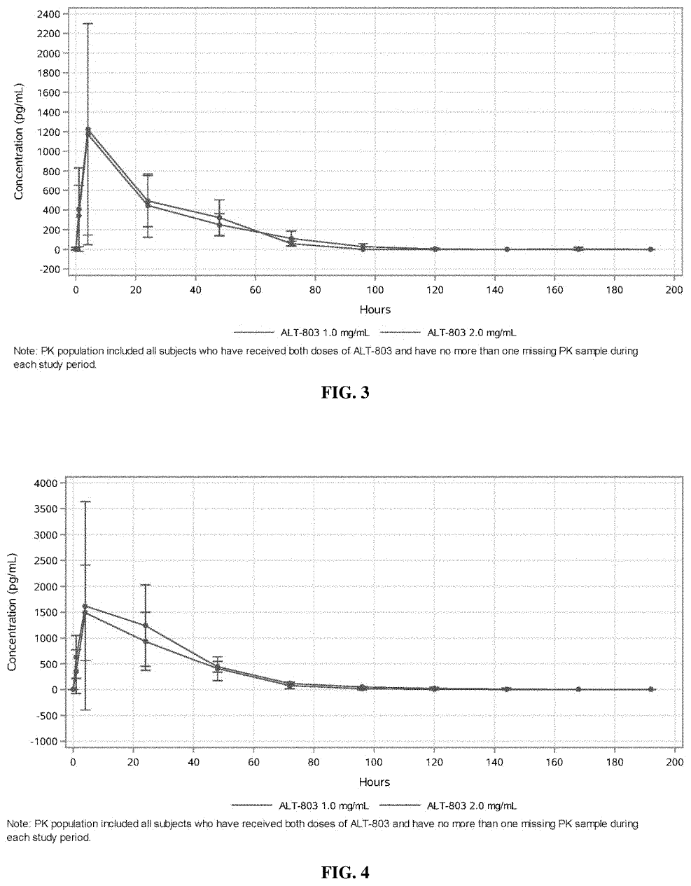

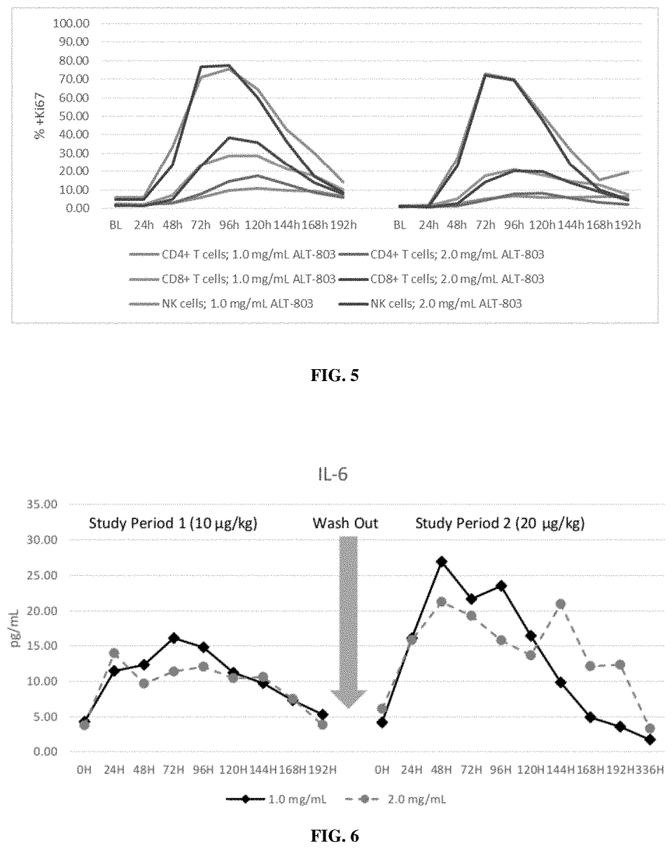

Mean serum ALT-803 concentration versus time profiles following administration of 10 μg/kg (Study Period 1) and 20 μg/kg (Study Period 2) are shown in FIG. 3 and FIG. 4. Serum ALT-803 concentrations appeared to increase until maximum concentration was achieved and decreased multi-exponentially following the absorption phase.

The mean Tₘₐₓ during Study Period 1 was approximately 9.71 hours for both dosing concentrations. Mean Cₘₐₓ was reported as 1219.17 and 1272.37 pg/mL for 1.0 and 2.0 mg/mL concentration, respectively at a dose of 10 μg/kg during Study Period 1. The T_{1/2} was approximately 28.68 and 14.87 hours for 1.0 and 2.0 mg/mL concentrations. AUC₀₋₇₂ was calculated as 1305.37 and 1561.73 day*pg/mL at 1.0 and 2.0 mg/mL concentrations. Similarly, AUC_{0-inf} was calculated as 1513.36 and 1813.22 day*pg/mL. Volume of distribution and Clearance for Study Period 1 were calculated as 1067448 and 459287 mL and Clearance was calculated as 669831 and 487914 mL/day at 1.0 and 2.0 mg/mL concentrations.

The mean Tₘₐₓ during Study Period 2 was approximately 17.33 and 4 hours for 1.0 and 2.0 dosing concentrations. Mean Cₘₐₓ was reported as 1899.91 and 1488.59 pg/mL for 1.0 and 2.0 mg/mL concentration, respectively at a dose of 2 μg/kg during Study Period 2. The T_{1/2} was approximately 19.48 and 15.66 hours for 1.0 and 2.0 mg/mL concentrations. AUC₀₋₇₂ was calculated as 2445.36 and 2349.35 day*pg/mL at 1.0 and 2.0 mg/mL concentrations. Similarly, AUC_{0-inf} was calculated as 7326.47 and 6562.51 day*pg/mL. Volume of distribution and Clearance for Study Period 2 were calculated as 288051 and 344495 mL and Clearance was calculated as 259977 and 310201 mL/day at 1.0 and 2.0 mg/mL concentrations. Estimated individual and mean PK parameters from this study are summarized in Table 3.

TABLE 3
Summary of PK Parameters (Population: PK Population)

| | Study Period 1 | | Study Period 2 | |
|---|---|---|---|---|
| | (10 μg/kg ALT-803) | | (20 μg/kg ALT-803) | |
| | ALT-803 | ALT-803 | ALT-803 | ALT-803 |
| | 1.0 mg/mL | 2.0 mg/mL | 1.0 mg/mL | 2.0 mg/mL |
| | n = 7 | n = 7 | n = 7 | n = 7 |
| Tmax (hr) | | | | |
| N | 7 | 7 | 6 | 7 |
| Mean | 9.71 | 9.71 | 17.33 | 4.00 |
| SD | 9.76 | 9.76 | 10.33 | 0.00 |
| Median | 4.00 | 4.00 | 24.00 | 4.00 |
| Min, Max | 4.00, 24.00 | 4.00, 24.00 | 4.00, 24.00 | 4.00, 24.00 |
| Cmax (pg/mL) | | | | |
| N | 7 | 7 | 6 | 7 |
| Mean | 1219.17 | 1272.37 | 1899.91 | 1488.59 |
| SD | 1102.71 | 1017.44 | 1930.17 | 921.12 |
| Median | 901.01 | 1048.86 | 1122.05 | 1463.10 |
| Min, Max | 208.58, 3310.50 | 301.92, 2843.30 | 711.65, 5719.75 | 587.97, 2823.00 |
| T½ (hr) | | | | |
| N | 5 | 5 | 6 | 5 |
| Mean | 28.68 | 14.87 | 19.48 | 15.66 |
| SD | 20.57 | 4.01 | 6.70 | 5.03 |
| Median | 20.67 | 14.86 | 19.25 | 13.29 |
| Min, Max | 16.16, 65.27 | 10.16, 20.44 | 12.32, 29.17 | 11.15, 22.75 |
| AUC0-72 (day*pg/mL) | | | | |
| N | 7 | 6 | 6 | 4 |
| Mean | 1305.37 | 1561.73 | 2445.36 | 2349.35 |
| SD | 786.11 | 887.71 | 1571.68 | 1120.86 |
| Median | 1043.29 | 1362.68 | 1943.94 | 2272.87 |
| Min, Max | 491.38, 2736.18 | 547.72, 2933.47 | 895.72, 5304.57 | 1126.82, 3724.84 |
| AUC0-inf (day*pg/mL) | | | | |
| N | 5 | 5 | 6 | 5 |
| Mean | 1513.36 | 1813.22 | 7326.47 | 6562.51 |
| SD | 921.99 | 830.73 | 4598.76 | 3724.54 |
| Median | 1336.39 | 1533.51 | 5067.98 | 5851.11 |
| Min, Max | 596.46, 2824.91 | 975.51, 3020.75 | 3736.46, 15316.75 | 2265.51, 12371.68 |
| Volume of Distribution | | | | |
| (mL) | | | | |
| N | 5 | 5 | 6 | 5 |
| Mean | 1067448 | 459287 | 288051 | 344495 |
| SD | 695283 | 326883 | 147783 | 388797 |
| Median | 1,014,352 | 457530 | 291409 | 161229 |
| Min, Max | 250961, 1879110 | 147906, 982494 | 96081, 511513 | 80203, 1026333 |
| Clearance (mL/day) | | | | |
| N | 5 | 5 | 6 | 5 |
| Mean | 669831 | 487914 | 259977 | 310201 |
| SD | 430551 | 242798 | 131661 | 251177 |
| Median | 478901 | 482552 | 276277 | 222856 |
| Min, Max | 258416, 1324484 | 165522, 799578 | 95320, 422860 | 119628, 750383 |

Pharmacokinetic Summary and Discussion:

In summary, the pharmacokinetic profiles for ALT-803 were well characterized and provided the following key findings:
ALT-803 concentrations reached the maximum concentrations at a Tₘₐₓ of approximately 4-24 days post the start of infusion.
The Cₘₐₓ and partial AUC's (AUC₀₋₇₂) appeared to increase in an approximately dose-dependent manner.
Half-life for Study Period 1 ranged from 10.16-65.27 days.
Half-life for Study Period 2 ranged from 11.15-29.17 days.
A comparison of PK parameters upon administration of subcutaneous injection(s) of 10 μg/kg and 20 μg/kg ALT-803 at concentrations of 1.0 mg/mL or 2.0 mg/mL did not indicate any difference.

Immune Cells:

Blood samples were collected for immune cell levels and activity, immune cell phenotype subsets, cytokine levels and immunogenicity testing, which include assays for anti-ALT-803 antibodies. Blood samples for immune cell levels and activity were collected at baseline (pre-dose), hour-24, hour-48, hour-72, hour-96, hour-120, hour-144, hour-168 and hour-192 for both Study Periods 1 (10 μg/kg ALT-803) and 2 (20 μg/kg ALT-803). Peripheral blood mononuclear cells (PBMCs) were isolated and subsequently batch analyzed by flow cytometric analysis.

The immune cell population included every subject for whom sufficient samples were collected to conduct the immunogenicity assay (N=20). All 20 subjects had samples collected and analyzed for all timepoints during Study Period 1. Five subjects were withdrawn from the study prior to Study Period 2 day 1 sample collection. One subject was withdrawn from Study Period 2 prior to the end of study sample collections. Therefore, fourteen subjects had samples collected and analyzed during Study Period 2.

ALT-803 administration to healthy volunteers strongly induces proliferation of natural killer (NK) cells and to a lesser degree CD8⁺ and CD4⁺ T-cells. The concentration of ALT-803 (1 mg/mL vs. 2 mg/mL) does not appear to have an impact on induction of immune cell proliferation (FIG. 5).

Cytokine Levels:

Serum was frozen and later batch analyzed for cytokines. Interleukin-2 (IL-2), interleukin-4 (IL-4), interleukin-6 (IL-6), interleukin-10 (IL-10), tumor necrosis factor (TNF) and interferon gamma (IFN-γ) concentrations were determined using a qualified commercial cytometric bead array Th1/Th2 cytokine Kit II.

The cytokine population included every subject for whom sufficient samples were collected to conduct the immunogenicity assay (N=20). All 20 subjects had samples collected and analyzed for all timepoints during Study Period 1. Five subjects were withdrawn from the study prior to Study Period 2 day 1 sample collection. One subject was withdrawn from SP 2 prior to the end of study sample collections. Therefore, fourteen subjects had samples collected and analyzed during Study Period 2.

Blood samples were collected for immune cell levels and activity, immune cell phenotype subsets, cytokine levels and immunogenicity testing, which include assays for anti-ALT-803 antibodies. Blood samples for cytokine testing were collected at baseline (pre-dose), hour-24, hour-48, hour-72, hour-96, hour-120, hour-144, hour-168 and hour-192 for both Study Periods 1 (10 μg/kg ALT-803) and 2 (20 μg/kg ALT-803).

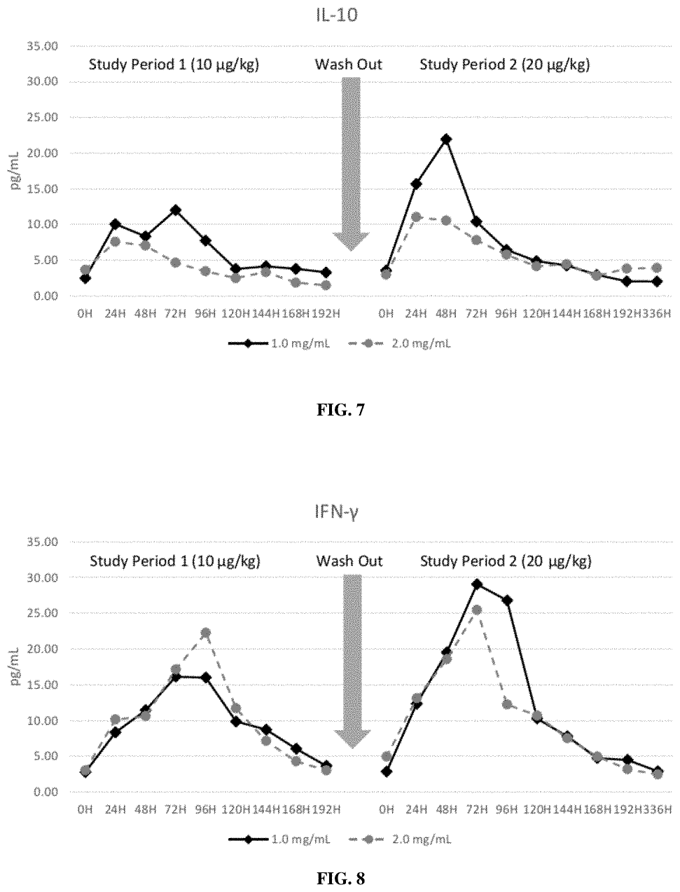

ALT-803 administration to healthy volunteers induces elevated serum levels of IL-6 (FIG. 6), IL-10 (FIG. 7) and IFN-γ (FIG. 8). The dose level of 20 μg/kg ALT-803 appears to induce higher serum levels of IL-6, IL-10 and IFN-γ compared to 10 μg/kg ALT-803. The concentration of ALT-803 (1 mg/mL vs. 2 mg/mL) does not appear to have an impact on cytokine induction.

No changes in serum levels of IL-2, IL-4 or TNF-α are observed following ALT-803 administration to healthy volunteers.

TABLE 4
Summary of General Health Questionnaire Data: Safety Population

| | ALT-803 | | ALT-803 | | All | |
|---|---|---|---|---|---|---|
| | 1.0 mg/mL | | 2.0 mg/mL | | Subjects | |
| | n = 10 | | n = 10 | | n = 20 | |
| Physical Functioning | | | | | | |
| Baseline, N | 10 | | 10 | | 20 | |
| Mean (SD) | 100.0 | (0.0) | 100.0 | (0.0) | 100.0 | (0.0) |
| End of Study, N | 6 | | 7 | | 13 | |
| Mean (SD) | 100.0 | (0.0) | 96.4 | (9.45) | 98.1 | (6.93) |
| Role limitations due to | | | | | | |
| physical health | | | | | | |
| Baseline, N | 10 | | 10 | | 20 | |
| Mean (SD) | 100.0 | (0.0) | 100.0 | (0.0) | 100.0 | (0.0) |
| End of Study, N | 6 | | 7 | | 13 | |
| Mean (SD) | 20.8 | (40.05) | 17.9 | (31.34) | 19.2 | (34.09) |
| Role limitations due to | | | | | | |
| emotional problems | | | | | | |
| Baseline, N | 10 | | 10 | | 20 | |
| Mean (SD) | 100.0 | (0.0) | 100.0 | (0.0) | 100.0 | (0.0) |
| End of Study, N | 6 | | 7 | | 13 | |
| Mean (SD) | 83.3 | (40.82) | 100.0 | (0.0) | 92.3 | (27.74) |
| Energy/Fatigue | | | | | | |
| Baseline, N | 10 | | 10 | | 20 | |
| Mean (SD) | 96.0 | (8.76) | 98.5 | (3.37) | 97.3 | (6.58) |
| End of Study, N | 6 | | 7 | | 13 | |
| Mean (SD) | 55.8 | (20.35) | 42.1 | (18.68) | 48.5 | (19.94) |
| Emotional well-being | | | | | | |
| Baseline, N | 10 | | 10 | | 20 | |
| Mean (SD) | 98.0 | (5.08) | 99.6 | (1.26) | 98.8 | (3.69) |
| End of Study, N | 6 | | 7 | | 13 | |
| Mean (SD) | 83.3 | (12.50) | 86.9 | (11.25) | 85.2 | (11.48) |
| Social Functioning | | | | | | |
| Baseline, N | 10 | | 10 | | 20 | |
| Mean (SD) | 100.0 | (0.0) | 100.0 | (0.0) | 100.0 | (0.0) |
| End of Study, N | 6 | | 7 | | 13 | |
| Mean (SD) | 60.4 | (18.40) | 66.1 | (27.68) | 63.5 | (23.08) |
| Pain | | | | | | |
| Baseline, N | 10 | | 10 | | 20 | |
| Mean (SD) | 100.0 | (0.0) | 100.0 | (0.0) | 100.0 | (0.0) |
| End of Study, N | 6 | | 7 | | 13 | |
| Mean (SD) | 44.6 | (26.85) | 35.4 | (18.62) | 39.6 | (22.29) |
| General Health | | | | | | |
| Baseline, N | 10 | | 10 | | 20 | |
| Mean (SD) | 96.0 | (4.59) | 98.0 | (3.50) | 97.0 | (4.10) |
| End of Study, N | 6 | | 7 | | 13 | |
| Mean (SD) | 77.5 | (12.94) | 90.7 | (8.86) | 84.6 | (12.49) |
| Health Change | | | | | | |
| Baseline, N | 10 | | 10 | | 20 | |
| Mean (SD) | 50.0 | (0.0) | 60.0 | (21.08) | 55.0 | (15.39) |
| End of Study, N | 6 | | 7 | | 13 | |
| Mean (SD) | 50.0 | (0.0) | 39.3 | (19.67) | 44.2 | (14.98) |

### CONCLUSIONS

ALT-803 administration to healthy volunteers strongly induced proliferation of natural killer (NK) cells and to a lesser degree CD8⁺ and CD4⁺ T-cells. Further, ALT-803 induced elevated serum levels of IL-6, IL-10 and IFN-γ. The dose level of 20 μg/kg ALT-803 appears to have induced higher serum levels of IL-6, IL-10 and IFN-γ compared to 10 μg/kg ALT-803. The concentration of ALT-803 (1 mg/mL vs. 2 mg/mL) did not appear to have an impact on immune cell proliferation or cytokine induction.

Clinical Laboratory Evaluation:

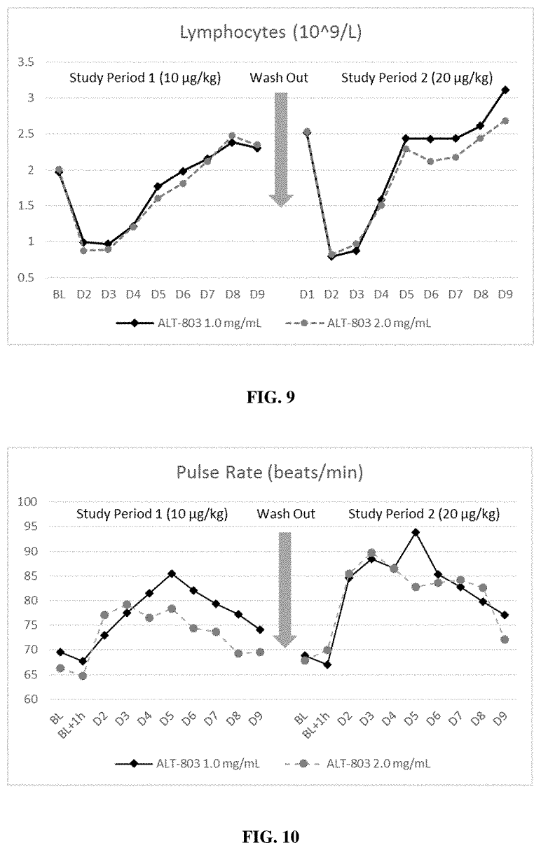

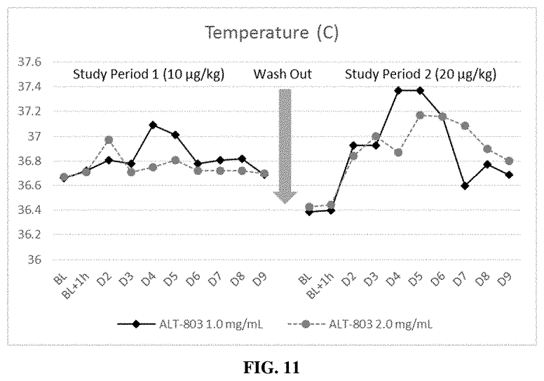

Blood samples were collected for a hematology panel evaluation, including basophils, basophils/leukocytes (%), eosinophils, eosinophils/leukocytes (%), mean corpuscular hemoglobin, mean corpuscular hemoglobin concentration, mean corpuscular volume, erythrocytes, erythrocyte distribution width, hematocrit, hemoglobin, leukocytes, lymphocytes, lymphocytes/leukocytes (%), mean platelet volume, monocytes, monocytes/leukocytes (%), neutrophils, neutrophils/leukocytes (%), platelets. Values for all chemistry assessments remained within normal ranges and were similar between ALT-803 concentrations and Study Periods. Of note, a significant drop in lymphocyte count was observed after both doses and both concentrations of ALT-803 (FIG. 9). This drop was approximately 50% and occurred 24 hours after dose. Lymphocyte count appeared to normalize by 96 hours (Day 5) after the dose.

Blood samples were collected for a chemistry panel evaluation which included albumin, alanine aminotransferase (ALT), alkaline phosphatase (ALP), aspartate aminotransferase (AST), bilirubin, calcium, carbon dioxide, chloride, creatinine, glucose, phosphate, potassium, protein, sodium and urea nitrogen. Values for all chemistry assessments remained within normal ranges and were similar between ALT-803 concentrations and Study Periods.

Detection of Anti-ALT-803 Antibodies:

Blood samples were collected for immune cell levels and activity, immune cell phenotype subsets, cytokine levels, and immunogenicity testing. Testing included assays for anti-ALT-803 antibodies. Blood samples for immunogenicity testing were collected at baseline (pre-dose), Day 1 of Study Period 2 (pre-dose), and at the end of study visit (Day 15 of Study Period 2).

The Immunogenicity population included every subject for whom sufficient samples were collected to conduct the immunogenicity assay (N=15). Five subjects were withdrawn from the study prior to Study Period 2 day 1 sample collection. Consequently, their baseline samples were not evaluated. One subject was withdrawn from the study prior to the end of study sample collections. Therefore, only the baseline and Study Period 2 day 1 samples were evaluated.

Of the 10 subjects who received 1.0 mg ALT-803 per mL, seven met criteria for evaluation. None of the seven developed detectable anti-ALT-803 antibodies. Of the 10 subjects who received doses of 2.0 mg ALT-803 per mL, eight met criteria for evaluation and one (0137-067-019) developed detectable anti-ALT-803 antibodies at the end of study visit (titer of 104).

ALT-803 administration to healthy volunteers strongly induces proliferation of natural killer (NK) cells and to a lesser degree CD8⁺ and CD4⁺ T-cells. Further, ALT-803 induces elevated serum levels of IL-6, IL-10 and IFN-γ. The dose level of 20 μg/kg ALT-803 appears to induce higher serum levels of IL-6, IL-10 and IFN-γ compared to 10 μg/kg ALT-803. The concentration of ALT-803 (1 mg/mL vs. 2 mg/mL) does not appear to have an impact on immune cell proliferation or cytokine induction.

The most common (>50% subjects) treatment-emergent adverse events include injection site reaction, chills, pyrexia, lower abdominal pain and headache and feeling of body temperature change. No subject experienced a treatment-emergent grade 3 or higher adverse event and no subject experienced a treatment-emergent serious adverse event. All subjects experienced an injection site reaction. Symptoms associated with injection site reactions appear to be similar when comparing between 1.0 and 2.0 mg/mL concentrations of ALT-803.

Of the 10 subjects who received 1.0 mg/mL ALT-803, seven met criteria for evaluation and none of the seven developed detectable anti-ALT-803 antibodies. Of the 10 subjects who received 2.0 mg/mL ALT-803, eight met criteria for evaluation and one (0137-067-019) developed detectable anti-ALT-803 antibodies at the end of study visit (titer of 104).

While not the intent of the trial, data suggests and support comparable safety between 1.0 mg/mL and 2.0 mg/mL concentrations of ALT-803. In summary, the pharmacokinetic profiles for ALT-803 were well characterized and provided the following findings:
ALT-803 concentrations reached the maximum concentrations at a Tₘₐₓ of approximately 4-24 days post the start of infusion.
The Cₘₐₓ and partial AUC's (AUC₀₋₇₂) appeared to increase in an approximately dose-dependent manner.
Half-life for Study Period 1 ranged from 10.16-65.27 days.
Half-life for Study Period 2 ranged from 11.15-29.17 days. A
Comparison of PK parameters upon administration of subcutaneous injection(s) of 10 μg/kg and 20 μg/kg ALT-803 at concentrations of 1.0 mg/mL or 2.0 mg/mL did not indicate any difference.

### OTHER EMBODIMENTS

From the foregoing description, it will be apparent that variations and modifications may be made to the invention described herein to adopt it to various usages and conditions. Such embodiments are also within the scope of the following claims.

1. A method of treating lymphopenia in a subject in need thereof, the method comprising administering to the subject a composition comprising a therapeutically effective amount of ALT-803, wherein the subject is undergoing radiation therapy and wherein the ALT-803 is administered within 48 hours of the radiation therapy.

2-8. (canceled)

9. The method of claim 1, wherein the radiation therapy is 50 Gray or less over 5 weeks.

10. The method of claim 1, wherein the radiation therapy is less than 45 Gray over 5 weeks.

11. The method of claim 1, wherein the radiation therapy is less than 40 Gray over 5 weeks.

12. The method of claim 1, wherein the radiation therapy is less than 35 Gray over 5 weeks.

13. The method of claim 1, wherein the composition comprising a therapeutically effective amount of ALT-803 is administered on the same day as the radiation therapy.

14. The method of claim 1, wherein the composition comprising a therapeutically effective amount of ALT-803 is administered within 24 hours of the radiation therapy.

15. (canceled)

16. (canceled)

17. The method of claim 1, further comprising administering one or more chemotherapeutic agents, cytokine antagonists, cytokine receptor antagonists, cytokines, chemokines, growth factors, hpG-CSF, EPO, lymphopoietin, adoptive cell therapy, anti-viral agents, viral entry receptor antagonists, checkpoint inhibitors, adjuvants, or combinations thereof.

18. The method of claim 17, wherein the adoptive cell therapy is administered and comprises administration of lymphocytes, stem cells, or combinations thereof.

19. The method of claim 17, wherein the cytokines are administered and comprise T helper 1 (TH1)-inducing cytokines.

20. The method of claim 17, wherein the cytokines are administered and comprise T helper 2 (TH2)-inducing cytokines.

21. The method of claim 19, wherein the TH1-inducing cytokines comprise interleukin-2 (IL-2), IL-10, IL-12, IL-18, IL-27, tumor necrosis factor-alpha (TNFα), TNFβ, an interferon, or combinations thereof.

22. The method of claim 20, wherein the TH2-inducing cytokines comprise IL-4, IL-5, IL-6, IL-9, IL-13, IL-19, IL-25, IL-31, IL-33, an interferon, or combinations thereof.

23-40. (canceled)

41. A method of treating lymphopenia in a subject in need thereof, the method comprising administering to the subject a composition comprising a therapeutically effective amount of ALT-803, wherein the subject is undergoing oncological chemotherapy and wherein the ALT-803 is administered with 48 hours of the chemotherapy.

42. The method of claim 41, wherein the subject is suffering from a disease or disorder associated with chemotherapy-induced lymphopenia.

43. The method of claim 42, wherein the composition comprising a therapeutically effective amount of ALT-803 is administered on the same day as the chemotherapy.

44. The method of claim 42, wherein the composition comprising a therapeutically effective amount of ALT-803 is administered within 24 hours of the chemotherapy.

45. The method of claim 41, further comprising administering one or more chemotherapeutic agents, cytokine antagonists, cytokine receptor antagonists, cytokines, chemokines, growth factors, hpG-CSF, EPO, lymphopoietin, adoptive cell therapy, anti-viral agents, viral entry receptor antagonists, checkpoint inhibitors, adjuvants, or combinations thereof.

46. The method of claim 45, wherein the adoptive cell therapy is administered and comprises administration of lymphocytes, stem cells, or combinations thereof.

47. The method of claim 45, wherein the cytokines are administered and comprise T helper 1 (T_{H}1)-inducing cytokines.

48. The method of claim 45, wherein the cytokines are administered and comprise T helper 2 (T_{H}2)-inducing cytokines.

49. The method of claim 47, wherein the TH1-inducing cytokines comprise interleukin-2 (IL-2), IL-10, IL-12, IL-18, IL-27, tumor necrosis factor-alpha (TNFα), TNFβ, an interferon, or combinations thereof.

50. The method of claim 48, wherein the T_{H}2-inducing cytokines comprise IL-4, IL-5, IL-6, IL-9, IL-13, IL-19, IL-25, IL-31, IL-33, an interferon, or combinations thereof.

51. The method of claim 1, wherein the therapeutically effective amount of ALT-803 is between about 0.1 μg/kg and about 100 mg/kg body weight.

52. The method of claim 41, wherein the therapeutically effective amount of ALT-803 is between about 0.1 μg/kg and about 100 mg/kg body weight.
